(19) United States
(12) Patent Application Publication
(10) Pub. No.: US 2025/0128063 A1
(43) Pub. Date: Apr. 24, 2025

(54) Neuromodulation Device

(71) Applicant: Galvani Bioelectronics Limited, Stevenage (GB)

(72) Inventors: Margarita J. Vervoordeldonk, Maarssen (NL); Misha Luyer, Eindhoven (NL)

(21) Appl. No.: 18/682,265

(22) PCT Filed: Aug. 9, 2022
(86) PCT No.: PCT/GB2022/052071

§ 371 Date: Feb. 8, 2024

Related U.S. Application Data
(60) Provisional application No. 63/231,611, filed on Aug. 10, 2021.

(51) Int. Cl. A61N 1/36; A61N 1/05

(52) U.S. Cl. CPC A61N 1/3606; A61N 1/3616; A61N 1/0556

(57) ABSTRACT

Stimulation of the spleen and/or neural activity of one or more nerves supplying the spleen, such as a nerve associated with the splenic arterial neurovascular bundle, can decrease C-Reactive Protein (CRP) levels and post-operative surgical complications. The system for stimulating the spleen and/or neural activity of one or more nerves supplying the spleen for reducing post-operative surgical complications can include at least one transducer in signaling contact with the spleen and/or the one or more nerves; and at least one controller electrically coupled to the at least one transducer. The at least one controller can be configured to control the operation of the at least one transducer to apply a stimulating signal to the spleen and/or the one or more nerves. The stimulating signal may produce a change in a physiological parameter indicative of a target engagement of the nerves resulting in a reduction in C-reactive protein (CRP) plasma levels and/or Interleukin-6 (IL-6) plasma levels after surgery and reducing post-operative surgical complications and/or days in hospital.

### FIELD OF THE INVENTION

The invention relates to neuromodulation of the spleen and/or one or more nerves supplying the spleen, more particularly to devices, systems and methods that stimulate neural activity in the spleen and/or nerve in an intra-operative setting. The invention also relates to devices, systems and methods that stimulate neural activity in the spleen and/or the one or more nerves supplying the spleen for reducing post-operative surgical complications.

### BACKGROUND

Discoveries in the field of neuroscience have shown how the autonomic nervous system (ANS) is involved in controlling the immune functions. This phenomenon is now commonly known as the “inflammatory reflex” (Tracey K J. Nature (2002); Koopman F A J Intern Med. 2017). Extensive evidence exists demonstrating that the efferent arm of the “inflammatory reflex” controls systemic immune responses via nerves going to the spleen (Huston J M J Exp Med (2006); Rosas-Ballina M Proc Natl Acad Sci USA (2008)). The spleen is innervated by the splenic nerves (Verlinden T J M Brain Behav Immun (2019)). This neuronal plexus runs along the splenic artery, together forming a neurovascular bundle, until it enters the splenic parenchyma where it releases neurotransmitters, in particular catecholamines, which subsequently modulate immune cells (Swirski F K Science (2009)). Exogenous electrical activation of neural pathways targeting the spleen, either via upstream activation of the cervical vagus nerve, or near end-organ activation of a splenic nerves, has been shown to induce cytokine modulation in small animals (Borovikova L V Nature (2000), Vida J Immunol (2011), Guyot B B I (2019)). Using acute rodent inflammatory models, this immunomodulatory effect has typically been demonstrated as a cytokine response, particularly reduction of tumor necrosis factor, which was mediated by noradrenaline.

Acute activation of the splenic nerves, using a commercially available cuff, reduced the production of tumor necrosis factor (TNF) and Interleukin-6 (IL-6) serum levels following endotoxemia in terminally anesthetized pigs (Donegà et al. PNAS 2021 118 (20)). In addition, splenic nerve stimulation caused amplitude- and frequency-dependent changes in splenic artery blood flow (BF) and systemic mean arterial blood pressure (MABP), which are directly correlated to nerve activation.

To translate these findings to a treatment for humans, a new cuff electrode was developed that can accommodate the pulsating features of the splenic artery and can effectively stimulate the splenic artery neurovascular bundle in humans.

Studies suggest that systemic inflammation after surgery has a negative impact on surgical outcomes in patients undergoing elective surgery. Postoperative C-reactive protein (CRP) levels have shown a strong correlation with complications in patients undergoing open versus laparoscopic colorectal surgery regardless of surgical approach (Straatman et al. Surgical 2018). CRP levels on the third postoperative day after major abdominal surgery were found to be predictive for major complications (Straatman et al. 2015). In gastric cancer patients undergoing gastrectomy, an association between perioperative interleukin 6 (IL-6) serum levels and post-operative morbidity was shown (Szczepanik et al. 2011). Plasma cytokine concentrations, including IL-6, on the first postoperative day have a predictive value on gastroesophageal anastomotic leakage at an early stage in patients undergoing esophagectomy (Song et al. 2017), often preceding an increase in CRP levels. Based on these findings, attenuation of an excessive inflammatory response within the perioperative period for surgical procedures, including esophagectomy, may reduce morbidity and mortality.

Thus, there is a need for further and improved ways of stimulating neural activity in the spleen and/or the one or more nerves supplying the spleen for treating inflammation and specifically reducing post-surgical complications. Generally, the post-surgical complications often occur in patients after major surgery and may induce a longer hospital stay are complications needing intervention (Clavien-Dindo classification ≥3), such as pneumonia, anastomotic leakage, and postoperative ileus. Postoperative complications are classified according to the Clavien-Dindo classification of surgical complications (see Appendix I-a; Dindo et al. 2004; Clavien et al. 2009). Cardiac complications include: cardiac arrest, cardiac ischemia/infarction, pericarditis, congestive heart failure and a-/dysrhythmias requiring intervention. Pulmonary complications include: (aspiration) pneumonia, pleural effusion/empyema, pneumothorax and atelectasis requiring intervention and acute respiratory distress syndrome and respiratory insufficiency requiring prolonged treatment or reintubation. Pneumonia is scored using the Uniform Pneumonia Score and anastomotic leakage is scored using the Esophagectomy Complications Consensus Group (ECCG) definition (see Appendices I-b; van der Sluis et al. 2014 and I-c; Low et al. 2015, respectively).

### SUMMARY

Splenic neural stimulation can offer a new therapeutic approach to lower the extent of inflammation-driven complications post surgery, both the number of complications and the severity of the complications.

Specifically, patients treated with stimulation of nerves supplying the spleen had C-reactive protein (CRP) plasma levels that were significantly reduced on post-operative days (POD) 2 and POD 3 following surgery compared to a matched control group. Specifically, the stimulation of nerves may include splenic arterial neurovascular bundle (NVB) stimulation. Reduced CRP levels may correlate with levels predicting a reduction in post-operative complications.

Thus, the invention provides a system for stimulating the neural activity of the spleen and/or one or more nerves supplying the spleen for reducing post-operative surgical complications, the system may comprise: at least one transducer or electrode in signaling contact with the nerve; and at least one controller electrically coupled to the at least one electrode. The at least one controller may be configured to control the operation of the at least one transducer or electrode to apply an electrical signal to the nerve. The electrical signal may produce a change in a physiological parameter indicative of a target engagement of the nerves resulting in a reduction in C-reactive protein (CRP) plasma levels and/or Interleukin-6 (IL-6) plasma levels after surgery and reducing post-operative surgical complications and/or days in hospital.

The electrical signal may comprise a plurality of pulses that may be biphasic and bipolar pulses. The plurality of pulses may comprise a pulse width ≥0.1 ms. The plurality of pulses may comprise a pulse width≤5 ms. The pulses may have a pulse width (of each phase) between 0.1 ms and 5 ms (this can be applicable to both positive and negative phases of the pulse, in the case of a biphasic pulse). The electrical signal may have a frequency of ≥0.1 Hz. The electrical signal may have a frequency of ≤300 Hz.

The system may further comprise a neural interface that includes the at least one transducer. The at least one transducer may include at least one electrode. The neural interface can be intravascular (e.g. catheter or stent) or extravascular (e.g. cuff or paddle). The neural interface may be placed around at least one splenic arterial nerve, placed around the splenic artery, placed on at least one splenic arterial nerve, placed on the splenic artery, placed into the splenic arterial nerve, or in proximity of the splenic arterial nerve. In certain favorable embodiments, the neural interface may comprise two electrically active electrode arms; an inert arm located in between each of the two electrically active electrode arms; and a spine that provides mechanical and electrical connection between the two electrically active electrode arms and the inert arm. The neural interface may be placed on the spleen. The neural interface may be a remote transducer.

The invention also provides a method of reversibly stimulating neural activity of the spleen and/or in one or more nerves supplying the spleen for reducing post-operative surgical complications, for example, wherein the nerve is associated with a neurovascular bundle (e.g. a splenic arterial nerve). The method comprises providing a system of the invention, positioning the at least one transducer or electrode in signaling contact with the spleen and/or splenic nerve, and controlling the operation of the least one transducer or electrode with at least one controller to apply an electrical signal to the spleen and/or the one or more nerves to stimulate neural activity. A remote transducer may be utilized for signaling contract and applying a signal to the spleen and/or the one or more nerves. The electrical signal may produce a change in a physiological parameter indicative of a target engagement of the spleen and/or the one or more nerves resulting in a reduction in C-reactive protein (CRP) plasma levels and/or Interleukin-6 (IL-6) plasma levels after surgery and reducing post-operative surgical complications and/or days in hospital.

These features, along with many others, are discussed in greater detail below.

### BRIEF DESCRIPTION OF THE DRAWINGS

Embodiments of the invention will be described, by way of example, with reference to the following drawings in which:

FIG. 1 illustrates a neural stimulation system.

FIG. 2 illustrates a wider system including the neural stimulation system from FIG. 1.

FIG. 3 illustrates a schematic presentation of a cuff electrode.

FIG. 4 shows an example presentation of an extension cable for connecting via a connector, to the cuff electrode from FIG. 3.

FIG. 5 illustrates a schematic presentation of another exemplary neural stimulation system.

FIG. 6 illustrates a schematic presentation showing the physiological relationship between the surgical-induced increase in CRP levels and the effect of neurostimulation.

FIG. 7 shows the percentage changes in splenic arterial (SpA) blood flow (BF) during 1-minute bipolar stimulation of the porcine SpA neurovascular bundle correlating with increasing amplitudes (FIG. 7A—4 mA; FIG. 7B—8 mA; FIG. 7C—12 mA; FIG. 7D 20 mA) and with evoked compound action potentials. (10 Hz, 0.4 mS PW (per phase), symmetric, biphasic square pulses, and increasing amplitudes (mA)).

FIG. 8 shows a correlation between evoked compound action potential (eCAP) and splenic arterial (SpA) myocardial blood flow (mBF) is 1:1. Each amplitude is showing the results from at least three independent stimulations.

FIG. 9 shows a correlation between velocity time integral (VTI) and transit time flow (TTF) values obtained during splenic arterial (SpA) stimulation. Stimulations of 10 Hz, 0.4 mS PW, symmetric, biphasic square pulses with increasing amplitudes ranging from 10 to 22 mA were given, (P<0.0001).

FIGS. 10A, 10B, and 10C shows fifty consecutive splenic arterial (SpA) stimulations and how these stimulations do not affect the activation profile in relation to blood flow change or evoked compound action potential (eCAP).

FIG. 11 shows blood flow changes in the splenic artery in individual patients in relation to the stimulation doses (mA) given. Run-in patients were used to optimize techniques including cuff placement and Doppler flow probe positioning. BF data from patient 2 were lost due to a technical error. Patient 5 was consented but not implanted due to COVID-19 pandemic restrictions.

FIG. 12 shows representative pictures acquired by the Doppler flow probe at baselined (FIG. 12A) and 15 seconds (FIG. 12B) after start of stimulation (maximal effect on blood flow (BF)).

FIG. 13 shows the dose dependent decrease in splenic arterial (SpA) blood flow (BF) and the stimulation amplitudes.

FIG. 14 shows how splenic arterial (SpA) neurovascular bundle (NVB) stimulation in patients undergoing minimal invasive esophagectomy-Ivor Lewis (MIE-LI) decreases C-reaction protein levels on postoperative day (POD) 2 and POD 3 between patients that underwent SpA NVB stimulation (n=13) [TRIAL], a Prosperity Score Matched (PSM) control group (n=13) [PSM]; and patients (n=65) that underwent MIE-LI in the same hospital in 2019-2020 receiving the same surgery and post-operative care [2019-2020].

FIG. 15 shows how splenic arterial (SpA) neurovascular bundle (NVB) stimulation in patients undergoing minimal invasive esophagectomy-Ivor Lewis (MIE-LI) does not affect leukocytes counts in the blood on postoperative day (POD) 2 and POD 3 between patients that underwent SpA NVB stimulation (n=13) [TRIAL], a Prosperity Score Matched (PSM) control group (n=13) [PSM]; and patients (n=65) that underwent MIE-LI in the same hospital in 2019-2020 receiving the same surgery and post-operative care [2019-2020].

The reader is advised that the attached drawings are not necessarily drawn to scale.

### DETAILED DESCRIPTION OF THE INVENTION

In the following description of various examples of the invention, reference is made to the accompanying drawings, which form a part hereof, and in which are shown by way of illustration various example structures, systems, and steps in which aspects of the invention may be practiced. It is to be understood that other specific arrangements of parts, structures, example devices, systems, and steps may be utilized and structural and functional modifications may be made without departing from the scope of the present invention. Also, while the terms “top,” “bottom,” “front,” “back,” “side,” and the like may be used in this specification to describe various example features and elements of the invention, these terms are used herein as a matter of convenience, e.g., based on the example orientations shown in the figures. Nothing in this specification should be construed as requiring a specific three-dimensional orientation of structures in order to fall within the scope of this invention.

The autonomic nervous system is an important regulator of local and systemic inflammation. The splenic anti-inflammatory circuits involve both the parasympathetic and the sympathetic nervous system. In the present invention, near end-organ stimulation, e.g. splenic neural stimulation (e.g., the spleen and/or one or more nerves or arterial neurovascular bundle) is performed as a means to reduce acute inflammation, e.g. intra-operative inflammation. The present invention provides data from a study in patients undergoing Minimally Invasive Ivor Lewis esophagectomy (MIE-IL) with laparoscopic cuff placement around the splenic arterial neurovascular bundle (SpA NVB) followed by stimulation and subsequent cuff removal. The present invention provides an unexpected therapeutic application based in part on the observation that CRP plasma levels were significantly reduced on Day 2 and 3 following surgery compared to a matched control group. Further, the findings provided that these reduced CRP levels correlate with levels predicting a reduction in post-operative complications. These findings suggest SpA NVB stimulation may offer a new therapeutic approach to lower the extent of inflammation-driven complications post surgery.

### The Spleen and Nerves Supplying the Spleen

The nerves supplying the spleen includes nerves directly supplying the spleen, e.g., splenic nerve and nerves indirectly supplying the spleen, e.g., vagus nerve. “Nerve” herein referenced throughout may refer to one or more nerves, any nerve supplying the spleen, or a splenic nerve directly innervating the spleen, e.g. nerves of splenic arterial neurovascular bundle. Innervation of the spleen is primarily sympathetic or noradrenergic, with peptide neurons likely representing the bulk of the remaining neurons. The splenic artery is covered with nervous tissue, which is derived from the coeliac plexus and continues with the splenic artery to the spleen as the splenic plexus. The splenic plexus enters the spleen at the hilum where the splenic artery diverges in terminal branches and the splenic plexus continues with these branches into the parenchyma of the spleen.

The splenic plexus includes several nerve fascicles which circumvent the main splenic artery from celiac artery to spleen, each nerve fascicle comprising a small bundle of nerve fibers. A nerve fascicle (or known as a peri-arterial nerve fascicle) that circumvents the splenic nerve is referred to herein as a splenic arterial nerve.

Embodiments of the invention involve applying an electrical signal or other means of stimulation to, and thereby modulating the neural activity of, the spleen and/or one or more nerves supplying the spleen. Additional embodiments involve applying an electrical signal or other means of stimulation to, and thereby modulating the neural activity of, one or more nerves supplying the spleen, wherein the nerve is associated with a neurovascular bundle. Alternate embodiments could apply to providing ultrasound to the spleen and/or one or more nerves supplying the spleen. Favorably, the nerve is a splenic arterial nerve.

In some embodiments, the nerve is a sympathetic nerve.

In some embodiments, the invention may involve applying a signal or other means of stimulation to one splenic arterial nerve. In other embodiments, the invention may involve a plurality (e.g., a bundle) of nerves, for example, a neurovascular bundle.

In other embodiments, the invention may involve applying an electrical signal or other means of stimulation to at least one splenic arterial nerve and the splenic artery. In other embodiments, the invention may involve applying an electrical signal or other means of stimulation to all splenic arterial nerves and the splenic artery.

Stimulation of the Spleen and/or One or More Nerves Supplying the Spleen

The invention involves applying a stimulation signal to the spleen and/or one or more nerves supplying the spleen. In an embodiment, the nerve is associated with a neurovascular bundle innervating the spleen (e.g. a splenic arterial nerve) to stimulate neural activity in the nerve. Stimulation refers to where signaling activity at least part of the nerve is increased compared to baseline neural activity in that part of the nerve, where baseline neural activity is the signaling activity of the nerve in the subject prior to any intervention. Put another way, stimulation results in the creation of neural activity which increases the total neural activity in that part of the nerve. Stimulation of the spleen and/or one or more nerves supplying the spleen may result in a change in splenic blood flow. The stimulation signal may be an electrical signal. The stimulation signal may also be an ultrasonic signal. Other stimulations signals may be applied that create neural actively on the spleen and/or the one or more nerves.

“Neural activity” of a nerve refers to the signaling activity of the nerve, for example the amplitude, frequency and/or pattern of action potentials in the nerve. The term “pattern,” as used herein in the context of action potentials in the nerve, is intended to include one or more of: local field potential(s), compound action potential(s), aggregate action potential(s), and also magnitudes, frequencies, areas under the curve and other patterns of action potentials in the nerve or sub-groups (e.g. fascicules) of neurons therein.

Stimulation typically involves increasing neural activity e.g. generating action potentials beyond the point of the stimulation in at least a part of the nerve. At any point along the axon, a functioning nerve will have a distribution of potassium and sodium ions across the nerve membrane. The distribution at one point along the axon determines the electrical membrane potential of the axon at that point, which in turn influences the distribution of potassium and sodium ions at an adjacent point, which in turn determines the electrical membrane potential of the axon at that point, and so on. This is a nerve operating in its normal state, wherein action potentials propagate from point to adjacent point along the axon, and which can be observed using conventional experimentation.

One way of characterizing a stimulation of neural activity is a distribution of potassium and sodium ions at one or more points in the axon, which is created not by virtue of the electrical membrane potential at adjacent a point or points of the nerve as a result of a propagating action potential, but by virtue of the application of a temporary external electrical field. The temporary external electrical field artificially modifies the distribution of potassium and sodium ions within a point in the nerve, causing depolarization of the nerve membrane that would not otherwise occur. The depolarization of the nerve membrane caused by the temporary external electrical field generates de novo action potential across that point. This is a nerve operating in a disrupted state, which can be observed by a distribution of potassium and sodium ions at a point in the axon (the point which has been stimulated) that has an electrical membrane potential that is not influenced or determined by the electrical membrane potential of an adjacent point.

Stimulation of neural activity is thus understood to be increasing neural activity from continuing past the point of signal application. Thus, the nerve at the point of signal application is modified in that the nerve membrane is reversibly depolarized by an electric field, such that a de novo action potential is generated and propagates through the modified nerve. Hence, the nerve at the point of signal application is modified in that a de novo action potential is generated.

When the signal is an electrical signal, the stimulation is based on the influence of electrical currents (e.g. charged particles, which may be one or more electrons in an electrode in signaling contact with the nerve, or one or more ions outside the nerve or within the nerve, for instance) on the distribution of ions across the nerve membrane.

Stimulation of neural activity encompasses full stimulation of neural activity in the nerve—that is, embodiments where the total neural activity is increased in the whole nerve.

Stimulation of neural activity may be partial stimulation. Partial stimulation may be such that the total signaling activity of the whole nerve is partially increased, or that the total signaling activity of a subset of nerve fibers of the nerve is fully increased (i.e. there is no neural activity in that subset of fibers of the nerve), or that the total signaling of a subset of nerve fibers of the nerve is partially increased compared to baseline neural activity in that subset of fibers of the nerve. For example, an increase in neural activity of ≤5%, ≤10%, ≤15%, ≤20%, ≤25%, ≤30%, ≤35%, ≤40%, ≤45%, ≤50%, ≤60%, ≤70%, ≤80%, ≤90% or ≤95%, or an increase of neural activity in a subset of nerve fibers of the nerve. For example, an increase in neural activity of ≥5%, ≥10%, ≥15%, ≥20%, ≥25%, ≥30%, ≥35%, ≥40%, ≥45%, ≥50%, ≥60%, ≥70%, ≥80%, ≥90% or ≥95%, or an increase of neural activity in a subset of nerve fibers of the nerve. Any combination of the upper and lower limits above is also possible. Neural activity may be measured by methods known in the art, for example, by the number of action potentials which propagate through the axon and/or the amplitude of the local field potential reflecting the summed activity of the action potentials.

Stimulation of neural activity may be an alteration in the pattern of action potentials. It will be appreciated that the pattern of action potentials can be modulated without necessarily changing the overall frequency or amplitude. For example, stimulation of neural activity may be (at least partially) corrective. As used herein, “corrective” is taken to mean that the modulated neural activity alters the neural activity towards the pattern of neural activity in a healthy subject, and this is called axonal modulation therapy. That is, upon cessation of signal application, neural activity in the nerve more closely resembles (ideally, substantially fully resembles) the pattern of action potentials in the nerve observed in a healthy subject than prior to signal application. Such corrective stimulation can be any stimulation as defined herein.

For example, application of a signal may result in an increase on neural activity, and upon cessation of signal application the pattern of action potentials in the nerve resembles the pattern of action potentials observed in a healthy subject. By way of further example, application of the signal may result in neural activity resembling the pattern of action potentials observed in a healthy subject and, upon cessation of the signal, the pattern of action potentials in the nerve remains the pattern of action potentials observed in a healthy subject.

Stimulation of neural activity may comprise altering the neural activity in various other ways, for example increasing a particular part of the baseline neural activity and/or stimulating new elements of activity, for example: in particular intervals of time, in particular frequency bands, according to particular patterns and so forth.

One advantage of the invention is that stimulation of neural activity is reversible. Hence, the modulation of neural activity is not permanent. For example, upon cessation of the application of a signal, neural activity in the nerve returns substantially towards baseline neural activity within 1-60 seconds, or within 1-60 minutes, or within 1-24 hours (e.g. within 1-12 hours, 1-6 hours, 1-4 hours, 1-2 hours), or within 1-7 days (e.g. 1-4 days, 1-2 days). In some instances of reversible stimulation, the neural activity returns substantially fully to baseline neural activity. That is, the neural activity following cessation of the application of a signal is substantially the same as the neural activity prior to a signal being applied. Hence, the nerve or the portion of the nerve has regained its normal physiological capacity to propagate action potentials.

The neural activity in an intra-operative or acute setting may lead to achieving the reduction in CRP/improvement in clinical outcome using stimulation of short duration (in time) or low repetition, e.g., 1×, 2×, 3×.

Patients treated with stimulation of the spleen and/or one or more nerves may be provided a new therapeutic approach to lower the severity of inflammation-driven complications post surgery. Patients treated with stimulation of the spleen and/or one or more nerves may also be provided a new therapeutic approach to reduce the number of days in hospital post-surgery. Patients treated with stimulation of the spleen and/or one or more nerves may also be provided a new therapeutic approach to increase the survival rate post-surgery. The one or more nerves may be nerves directly supplying the spleen, e.g., splenic nerve, or nerves indirectly supplying the spleen, e.g., vagus nerve, any nerve innervating the spleen, or a splenic nerve directly innervating the spleen, e.g. nerves of splenic arterial neurovascular bundle.

Nerve and/or splenic arterial (SpA) neurovascular bundle (NVB) stimulation may offer a new therapeutic approach increase the survival rate post-surgery.

In other embodiments, stimulation of neural activity may be substantially persistent. As used herein, “persistent” is taken to mean that the neural activity has a prolonged effect. For example, upon cessation of the application of a signal, neural activity in the nerve remains substantially the same as when the signal was being applied—i.e. the neural activity during and following signal application is substantially the same. Reversible modulation is preferred.

### Suitable Forms of a Stimulation Signal

The invention uses stimulation of the spleen and/or one or more nerves supplying the spleen. In an embodiment, the stimulation may be ultrasound. In another embodiment, the stimulation may be propagated as an electrical signal in the spleen and/or the one or more nerves. In an alternate embodiment, the stimulation may be an electrical signal applied via at least one transducer in signaling contact with the spleen and/or one or more nerves supplying the spleen. The transducer(s) may be in physical contact with the spleen and/or the one or more nerves, such as an intentional signal applied to the spleen and/or a splenic nerve. The transducer(s) may also be a remote transducer not in physical contact with the spleen and/or the one or more nerves supplying the spleen. The at least one transducer may include at least one electrode placed in signaling contact with the spleen and/or the one or more nerves supplying the spleen. The one or more nerves may be associated with a neurovascular bundle (e.g. a splenic arterial nerve). As used herein, “signaling contact” is where at least part of the signal applied to the spleen and/or or more nerves supplying the spleen is received at the spleen and/or the one or more nerves supplying the spleen. Signaling contact may be applied via at least one transducer and/or electrode. Signaling contact may be applied via at least one transducer and/or electrode in physical contact with the spleen and/or one or more nerves supplying the spleen. Signaling contact may be achieved by an ultrasonic signal.

Stimulation signals applied according to the invention are ideally non-destructive. As used herein, a “non-destructive signal” is a signal that, when applied, does not irreversibly damage the underlying neural signal conduction ability of the nerve. That is, application of a non-destructive signal maintains the ability of the nerve or fibers thereof, or other nerve tissue to which the signal is applied, to conduct action potentials when application of the signal ceases, even if that conduction is in practice artificially stimulated as a result of application of the non-destructive signal. A stimulation signal can be an electrical signal. Alternatively, a stimulation signal can be an ultrasonic signal.

Electrical signals applied according to the invention may be voltage or current controlled.

The electrical signal may be characterized by one or more electrical signal parameters. The electrical signal parameters include waveform, frequency, charge, charge density, pulse width, interphase interval, duty cycle, and amplitude.

Alternatively or additionally, the electrical signal may be characterized by the pattern of application of the electrical signal to the nerve. The pattern of application refers to the timing of the application of the electrical signal to the nerve. The pattern of application may be continuous application or periodic application, and/or episodic application.

Episodic application refers to where the electrical signal is applied to the nerve for a discrete number of episodes throughout a day. Each episode may be defined by a set duration or a set number of iterations of the electrical signal, as described in more detail below.

Continuous application refers to where the electrical signal is applied to the nerve in a continuous manner. Where the electrical signal is applied continuously and episodically, it means that the signal is applied in a continuous manner for each episode of application. In embodiments where the electrical signal is a series of pulses, the gaps between those pulses (i.e. between the pulse width and the phase duration) do not mean the signal is not continuously applied.

Periodic application refers to where the electrical signal is applied to the nerve in a repeating pattern (e.g. an on-off pattern). Where the electrical signal is applied periodically and episodically, it means that the signal is applied in a periodic manner for each episode of application.

Electrical signal parameters and patterns of signal application for stimulating neural activity in the spleen and/or one or more nerves supplying the spleen are disclosed. The stimulation leads to reduced post-surgical inflammatory markers, including CRP, and/or wherein stimulations lead to reduced post-surgical complication rates and days in hospital.

Favorable signal parameters and patterns of application are discussed in detail below.

### Waveform

Modulation (e.g. stimulation) of the spleen and/or one or more nerves supplying the spleen, wherein the nerve is associated with a neurovascular bundle (e.g. a splenic arterial nerve) can be achieved using electrical signals which serve to replicate the normal neural activity of the nerve. Thus, the waveform of the electrical signal comprises one or more pulses, each with a defined pulse width. The pulses are favorably rectangular pulses. The pulses may be square pulses. However, other pulse waveforms such as sawtooth, sinusoidal, triangular, trapezoidal, quasitrapezoidal or complex waveforms may also be used with the invention.

The pulses can be monophasic or biphasic in nature. The term “biphasic” refers to a pulse which applies to the nerve over time both a positive and negative charge (anodic and cathodic phases). In certain embodiments, the biphasic pulses can favorably be charge-balanced.

The pulses may be charge-balanced. A charge-balanced pulse refers to a pulse which, over the period of the pulse, applies equal amounts (or thereabouts) of positive and negative charge to the nerve.

The pulses may be symmetric or asymmetric. A symmetric pulse is a pulse where the waveform when applying a positive charge to the nerve is symmetrical to the waveform when applying a negative charge to the nerve. An asymmetric pulse is a pulse where the waveform when applying a positive charge to the nerve is not symmetrical with the waveform when applying a negative charge to the nerve.

The pulses may have a pulse width (of each phase) between 100 and 5000 μs (this can be applicable to both positive and negative phases of the pulse, in the case of a biphasic pulse). The pulses may have a pulse width (of each phase) between 100 and 3000 μs (this can be applicable to both positive and negative phases of the pulse, in the case of a biphasic pulse). The pulses may have a pulse width (of each phase) between 100 and 2000 μs, favorably between 400 and 1000 μs (this can be applicable to both positive and negative phases of the pulse, in the case of a biphasic pulse). In an additional embodiment, the pulse may have a pulse width (of each phase) between 400 and 1000 μs. For example, the pulse width may be ≤500 μs, ≤600 μs, ≤700 μs, ≤800 μs, ≤900 μs, ≤1000 μs, S 2000μ, ≤3000 μs, ≤4000 μs, or ≤5000 μs. Additionally or alternatively, the pulse width may be ≥100 μs, ≥200 μs, ≥300 μs, ≥400 μs, ≥500 μs, ≥600 μs, ≥700 μs, ≥800 μs, or ≥900 μs. Any combination of the upper and lower limits above is also possible. The pulse width may additionally be limited by the frequency.

A pulse width refers to a width (or time duration) of a primary phase of the waveform. In cases where a pulse comprises a first phase that is the primary phase and a second phase which is the recovery phase, for example an anodic and/or a cathodic phase, the pulse width refers to a width (or duration) of the first phase. Interphase interval refers to the time period from the end of a pulse to the start of the next pulse. In one favorable embodiment, the interphase delay is constant between all of the pulses of the pulse train. For example, the interphase delay may be between 0 and 300 μs. In another embodiment, the interphase delay may be between 0 and 200 μs.

If the biphasic pulse is asymmetric, but remains charged balanced, then the areas of the opposing phases must equal. Amplitude (see below) can be reduced, but the pulse width would need to be extended to ensure the area under the curve is matched.

In an exemplary embodiment, the waveform is a pulse train with biphasic, asymmetric, charge balanced square pulses. In another exemplary embodiment, the waveform is a pulse train with biphasic, asymmetric, charge balanced rectangular pulses.

### Amplitude

For the purpose of the invention, the amplitude is referred to herein in terms of charge density per phase. Charge applied to the nerve by the electrical signal is defined as the integral of the current over one phase (e.g. over one phase of the biphasic pulse in the case of a charge-balanced biphasic pulse). Thus, charge density per phase applied to the nerve by the electrical signal is the charge per phase per unit of contact area between at least one electrode and the nerve, and also the integral of the current density over one phase of the signal waveform. Put another way, the charge density per phase applied to the nerve by the electrical signal is the charge per phase applied to the nerve by the electrical signal divided by the contact area between at least one electrode (generally the cathode) and the nerve.

The charge density per phase required by the invention represents the amount of energy required to stimulate neural activity in the spleen and/or one or more nerves supplying the spleen, wherein the nerve is associated with a neurovascular bundle (e.g. a splenic arterial nerve) to increase immunosuppressive effects.

In an embodiment, the charge density per phase required to stimulate neural activity in a splenic arterial nerve may be between 5 μC to 150 μC per cm² per phase or in some cases between 5 μC to 180 μC per cm² per phase, or may be between 5 μC to 250 μC per cm² per phase, using an extra vascular cuff (values may be slightly affected by electrode design). For example, the charge density per phase applied by the electrical signal may be ≤10 μC per cm² per phase, ≤15 μC per cm² per phase, ≤20 μC per cm² per phase, ≤25 μC per cm² per phase, ≤30 μC per cm² per phase, ≤40 μC per cm² per phase, ≤50 μC per cm² per phase, ≤75 μC per cm² per phase, ≤100 μC per cm² per phase, ≤125 μC per cm² per phase, ≤150 μC per cm² per phase, ≤180 μC per cm² per phase, ≤200 μC per cm² per phase, ≤225 μC per cm² per phase, or, ≤260 μC per cm² per phase. Additionally or alternatively, the charge density per phase applied by the electrical signal may be ≥5 μC per cm² per phase, ≥10 μC per cm² per phase, ≥15 μC per cm² per phase, ≥20 μC per cm² per phase, ≥25 μC per cm² per phase, ≥30 μC per cm² per phase, ≥40 μC per cm² per phase, ≥50 μC per cm² per phase, ≥75 μC per cm² per phase, ≥100 μC per cm² per phase, ≥125 μC per cm² per phase, ≥150 μC per cm² per phase, ≥175 μC per cm² per phase, ≥200 μC per cm² per phase, or ≥225 μC per cm² per phase. Any combination of the upper and lower limits above is also possible.

The indicated estimation of charge density per phase required to stimulate neural activity in a human splenic arterial nerve may be between approximately ≥70 μC per cm² per phase-≤1300 μC per cm² per phase. For example, the charge density per phase applied by the electrical signal may be ≤80 μC per cm² per phase, ≤140 μC per cm² per phase, ≤170 μC per cm² per phase, ≤230 μC per cm² per phase, ≤250 μC per cm² per phase, ≤300 μC per cm² per phase, ≤350 μC per cm² per phase, ≤400 μC per cm² per phase, ≤450 μC per cm² per phase, ≤500 μC per cm² per phase, ≤1100 μC per cm² per phase, or ≤1300 μC per cm² per phase. Additionally or alternatively, the charge density per phase applied by the electrical signal may be ≥70 μC per cm² per phase, ≥140 μC per cm² per phase, ≥170 μC per cm² per phase, ≥230 μC per cm² per phase, ≥250 μC per cm² per phase, ≥300 μC per cm² per phase, ≥350 μC per cm² per phase, ≥400 μC per cm² per phase, ≥450 μC per cm² per phase, ≥500 μC per cm² per phase, ≥1100 μC per cm² per phase, or ≥1300 μC per cm² per phase. Any combination of the upper and lower limits above is also possible. The total charge applied to the nerve by the electrical signal in any given time period is a result of the charge density per phase of the signal, in addition to the frequency of the signal, the pattern of application of the signal and the area in contact between at least one electrode and the nerve. The frequency of the signal, the pattern of application of the signal and the area in contact between at least one electrode and the nerve are discussed further herein.

It will be appreciated by the skilled person that the amplitude of an applied electrical signal necessary to achieve the intended stimulation of the neural activity will depend upon the positioning of the electrode and the associated electrophysiological characteristics (e.g. impedance). It is within the ability of the skilled person to determine the appropriate current amplitude for achieving the intended modulation of the neural activity in a given subject. The charge density per phase for each stimulation may be constant and be the same value for the stimulating neural activity. In another embodiment, the charge density per phase for each stimulation may vary and be different values for the stimulating neural activity.

It would be of course understood in the art that the electrical signal applied to the nerve would be within clinical safety margins (e.g. suitable for maintaining nerve signaling function, suitable for maintaining nerve integrity, and suitable for maintaining the safety of the subject). The electrical parameters within the clinical safety margin would typically be determined by pre-clinical studies. Additional electrical parameters can easily be determined by those of ordinary skill in pre-clinical models.

### Episodic Application

Episodic application in intra-operative/acute applications refers to where the electrical signal is applied to the nerve for a discrete number of episodes occurring within the timeframe of the operation. This application could be either in short succession (as in the clinical trial) or during the beginning, middle, or end of the surgical procedure, or during any combination of these three timeframes (e.g. at the beginning and during the middle of the surgical procedure, at the beginning and end of the surgical procedure, during the middle and end of the surgical procedure, or at the beginning, during the middle and at the end of the surgical procedure).

In an embodiment, the electrical signal according to the invention may be applied in acute applications for a discrete number of episodes for a discrete time period over the timeframe of the operation. In a favorable embodiment, the stimulation may be applied one, two, or three times for 1 or 2 minutes in a 20-minute timeframe (e.g. 5 minutes in between), and then remove the lead. In another embodiment, the stimulation may be applied one, two, or three times for 5 minutes in a 30-minute timeframe (e.g. 5 minutes in between), and then remove the lead. In other embodiments, there may be 1, 2, 3, 4, or 5 stimulations for 1 or 2 minutes in a 30-minute timeframe, and then remove the lead. In another embodiment, the stimulation may be applied 1, 2, 3, 4 or five times for 1, 2, 3, 4 or 5 minutes in a 1-hour timeframe (e.g. 5 minutes in between), and then remove the lead. In a further embodiment, the stimulation may be applied 3, 4, or 5 times for 1, 2, 3, 4 or 5 minutes in a 1-hour timeframe (e.g. 5 minutes in between), and then remove the lead. Various combinations of stimulation times and operation times may be utilized for the intra-operative/acute applications. For each of the stimulations, the amplitude and/or charge density may remain constant throughout the stimulating neural activity. In another embodiment, the amplitude and/or charge density may vary (increasing/decreasing) for the stimulating neural activity, depending on, e.g. the SpA BF changes measured.

Each episode may be defined by a set duration or a set number of iterations of the electrical signal. In some embodiments, each episode comprises applying to the nerve between 30 and 10,000 pulses, e.g. between 30 and 3,000 pulses of the electrical signal, between 100 and 2,400 pulses of the electrical signal, between 200 and 1,200 pulses of the electrical signal, between 400 and 600 pulses of the electrical signal, etc. For example, each episode may comprise applying ≤400, ≤800, ≤1,200, ≤1,600, ≤2,000, ≤2,400, ≤3,000, ≤10,000 pulses of the electrical signal. In another example, each episode may comprise applying ≤200, ≤400, ≤600, ≤800, ≤1,000, or ≤1,200 pulses of the electrical signal. In a further example, each episode may comprise applying ≤400, ≤425, ≤450, ≤475, ≤500, ≤525, ≤550, ≤575, or ≤600 pulses of the electrical signal.

In other embodiments, each episode comprises between 20 and 40 iterations of the periodic pattern. For example, each episode comprises applying 20, 25, 30, 35, or 40 iterations of the periodic pattern, or any number therebetween. The higher the frequency, the lower the number of iterations.

### Periodic Application

Periodic application refers to where the electrical signal is applied to the nerve in a repeating pattern. In certain embodiments, the preferred repeating pattern is an on-off pattern, where the signal is applied is applied for a first duration, referred to herein as an ‘on’ duration, then stopped for a second duration, referred to herein as an ‘off’ duration, then applied again for the first duration, then stopped again for the second duration, etc.

The periodic on-off pattern can have an on duration of between 0.1 and 10 seconds and an off duration of between 0.5 and 30 seconds. For example, the on duration (referred as the time during which pulses at a certain frequency and amplitude are delivered to the nerve) may be ≤0.2 s, ≤0.5 s, ≤1 s, ≤2 s, ≤5 s, or ≤10 s. Alternatively or additionally, the on duration may be ≥0.1 s, ≥0.2 s, ≥0.5 s, ≥1 s, ≥2 s, or ≥5 s. Any combination of the upper and lower limits above for the on duration is also possible. For example, the off duration (referred to the time between on periods, during which no pulses are delivered to the nerve) may be ≤1 s, ≤3 s, ≤5 s, ≤10 s, ≤15 s, ≤20 s, ≤25 s, or ≤30 s. Alternatively or additionally, the off duration may be ≥0.5 s, ≥1 s, ≥2 s, ≥5 s, ≥10 s, ≥15 s, ≥20 s, or ≥25 s. Any combination of the upper and lower limits above for the off duration is also possible.

In an exemplary embodiment, the periodic on-off pattern has an on duration of 0.5 sec on, and 4.5 sec off. In another example, the periodic on-off pattern has an on duration of 0.5 s on, and 5 sec off for up to 10 Hz pulses. For frequency higher than 10 Hz (for example 30 Hz) an example periodic on-off pattern has an on duration of or 0.1 s on, and an off duration of 3 s. In other words, a ratio of the on duration to the off duration may be 1:5. In other favorable embodiments, the ratio can be 1:6, 1:7, 1:8, 1:9, 1:10, 1:20 or 1:30. A ratio of the on duration to the off duration may be 1:10 for pulse frequency up to 10 Hz, and a ratio of the on duration to the off duration may be 1:30 for pulse frequency higher than 10 Hz.

Where the electrical signal is applied periodically and episodically, it means that the signal is applied in a periodic manner for each episode of application.

Periodic application may also be referred to as a duty cycled application. A duty cycle represents the percentage of time that the signal is applied to the nerve for a cycle of the periodic pattern. For example, a duty cycle of 20% may represent a periodic pattern having an on duration of 2 seconds, and an off duration of 10 seconds. Alternatively, a duty cycle of 20% may represent a periodic pattern having a on duration of 1 seconds, and an off duration of 5 seconds. In other words, periodic application may also be referred to as on-off pattern stimulation, or burst stimulation.

Duty cycles suitable for the present invention are between 0.1% and 100%.

### Frequency

Frequency is defined as the reciprocal of the phase duration of the electrical waveform (i.e. 1/phase).

The frequencies for stimulating the spleen and/or one or more nerves supplying the spleen can be selected based on the choice of nerve to be stimulated. In an exemplary embodiment, the nerve is associated with a neurovascular bundle (e.g. a splenic arterial nerve), and the frequencies are selected to be favorable for stimulating a neurovascular bundle, e.g., of the splenic arterial nerve (as further detailed below). In particular, the stimulation may include preferred frequencies for embodiments where the electrical signal is applied periodically and for embodiments where the electrical signal is applied continuously.

As previously noted, embodiments where the electrical signal is applied periodically and embodiments where the electrical signal is applied continuously provide different functions using different stimulation parameters. In one embodiment, continuous stimulation can be used to induce blood flow changes within the splenic vasculature that can be detected and used as on-table or peri-surgically as an indicator of successful electrode placement and/or amplitude determination; and a periodic stimulation can be used as a favorable treatment paradigm, whereby such blood flow change and/or other possible systemic cardiovascular effects are minimized or avoided whilst maintaining efficacy as a treatment.

In embodiments where the electrical signal is applied periodically, the electrical signal has a frequency of: ≤300 Hz, ≤50 Hz, or ≤10 Hz. For example, the frequency of the electrical signal may be ≤50 Hz, ≤100 Hz, ≤150 Hz, ≤200 Hz, ≤250 Hz or ≤300 Hz. In other examples, the frequency of the electrical signal may be ≤10 Hz, ≤15 Hz, ≤20 Hz, ≤25 Hz, ≤30 Hz, ≤35 Hz, ≤40 Hz, ≤45 Hz, or ≤50 Hz. In further examples, the frequency may be ≤1 Hz, ≤2 Hz, ≤5 Hz, or ≤10 Hz. Additionally or alternatively, the frequency of the electrical signal may be ≥10 Hz, ≥15 Hz, ≥20 Hz, ≥25 Hz, ≥30 Hz, ≥35 Hz ≥40 Hz, ≥45 Hz, or ≥50 Hz. In other examples, the frequency of the electrical signal may be ≥0.1 Hz, ≥0.2 Hz, ≥0.5 Hz, ≥1 Hz, ≥2 Hz, or ≥5 Hz. Any combination of the upper and lower limits above is also possible.

In embodiments where the electrical signal is applied continuously, the electrical signal has a frequency of ≤50 Hz, ≤10 Hz, or ≤2 Hz, or ≤1 Hz. For example, the frequency may be ≤1 Hz, ≤2 Hz, ≤5 Hz, or ≤10 Hz. In other examples the frequency may be ≤0.1 Hz, ≤0.2 Hz, ≤0.3 Hz, ≤0.4 Hz≤0.5 Hz, ≤0.6 Hz≤0.7 Hz, ≤0.8 Hz, or ≤0.9 Hz. Additionally or alternatively, the frequency of the electrical signal may be ≥0.1 Hz, ≥0.2 Hz, ≥0.5 Hz, ≥1 Hz, ≥2 Hz, or ≥5 Hz. Any combination of the upper and lower limits above is also possible.

Where the signal waveform comprises a pulse train, the pulses are applied to the nerve at intervals according to the above-mentioned frequencies. For example, a frequency of 50 Hz results in 50 pulses being applied to the nerve per second.

### Electrode and Neural Interface Design

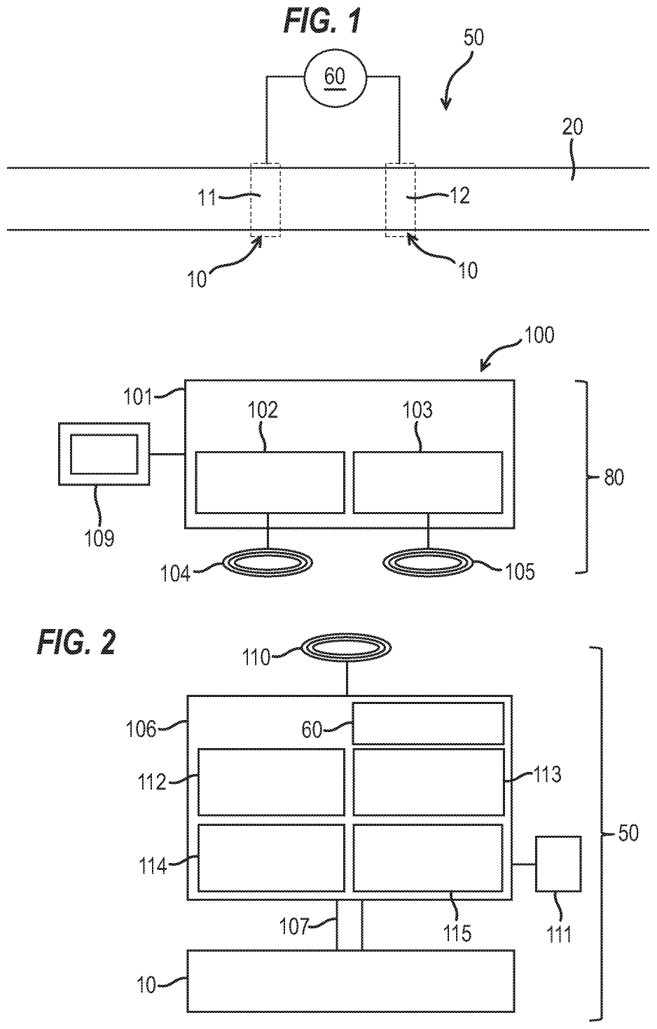

The electrical signal is applied to the spleen and/or one or more nerves supplying the spleen via at least one electrode in signaling contact with the nerve. As illustrated in FIG. 1, the at least one electrode may be positioned on a neural interface 10.

In some embodiments, the electrode and/or neural interface 10 may be configured for placement around at least one splenic arterial nerve and/or around the splenic artery. In such embodiments, the neural interface 10 may be a cuff type interface, but other interfaces which partially or fully circumvent (or encircle) the nerve may be used.

In other embodiments, the neural interface 10 may be configured for placement on the at least one splenic arterial nerve and/or on the splenic artery. In such embodiments, the neural interface 10 may be a patch or clip type interface.

In other embodiments, the neural interface 10 is configured for placement in and/or around the splenic artery. In such embodiments, the neural interface may be a catheter or a probe type interface. The neural interface 10 as a catheter or a probe type interface may comprise one or more of any of the catheter or probe type interfaces identified in PCT Patent Application Serial No. PCT/IB2019/060855, filed Dec. 16, 2019, and titled “Neurostimulation Device for Blocking Blood Flow Between Electrodes,” which application in its entirety is incorporated by reference herein.

In other embodiments, the neural interface 10 is configured for placement in at least one splenic arterial nerve. In such embodiments, the neural interface 10 may be a pin type interface. The neural interface 10 as a pin type interface may comprise one or more of any of the pin type interfaces or stent electrodes identified in PCT Patent Application Serial No. PCT/GB2020/053223, filed Dec. 15, 2020, and titled “Stent-Electrode Intervascular Neuromodulator and Associated Methods for Activation of a Nerve,” which application in its entirety is incorporated by reference herein.

The neural interface comprises at least one electrode. The electrodes may be fabricated from, or be partially or entirely coated with, a high charge capacity material such as platinum black, iridium oxide, titanium nitride, tantalum, poly(elthylenedioxythiophene) and suitable combinations thereof.

The at least one electrode may be a flat interface electrode which is flexible, particularly in embodiments where the neural interface is configured for placement in, on or around the at least one splenic arterial nerve and/or the splenic artery so as to circumvent the nerve, and/or the splenic artery when the neural interface 10 is secured on the nerve. However, other electrode types are also suitable for use in the invention.

Other electrode types suitable for the present invention include cuff electrodes (e.g. spiral cuff, helical cuff or flat interface); hemi-cuff electrodes; a mesh, a linear rod-shaped lead, paddle-style lead or disc contact electrode (including multi-disc contact electrodes); hook electrodes; sling electrodes; intrafascicular electrodes; glass suction electrodes; paddle electrode; and percutaneous cylindrical electrodes. Regardless of the exact design, the electrode/neural interface 10 should be easily applied and removed (e.g., during surgery) without damage to the nerve. The neural interface 10 as a cuff electrode may comprise one or more of any of the cuff electrodes identified in PCT Patent Application Serial No. PCT/GB2018/052076, filed Jul. 23, 2018, and titled “Electrode Devices for Neurostimulation;” PCT Patent Application Serial No. PCT/GB2018/052075, filed Jul. 23, 2018, and titled “Electrode Devices and Methods of Manufacturing;” and PCT Patent Application Serial No. PCT/GB2018/052074, filed Jul. 23, 2018, and titled “Electrode Devices for Neurostimulation,” which applications in their entirety are incorporated by reference herein.

FIG. 1 shows a schematic diagram of a neural stimulation system 50. The neural stimulation system 50 includes a neural interface 10 or an exemplary bipolar electrode configuration 10 wherein the electrodes are placed in signaling contact with at least one splenic arterial nerve and/or splenic artery 20. As illustrated in FIG. 1, the bipolar electrode configuration 10 may comprise a first electrode 11 and a second electrode 12, referred to herein as a bipolar electrode configuration 10. As explained elsewhere herein, suitable signaling contact may be achieved by placing the electrodes 11, 12 around (i.e. partially or fully circumventing) the nerve and/or artery 20, on the nerve and/or the artery 20, or in the splenic nerve 20, or in the artery 20.

As shown in the embodiment illustrated in FIG. 1, the first electrode 11 and second electrodes 12 are positioned along the longitudinal axis of the nerve 20. An electrical signal may be applied to the electrodes such that the first electrode 11 is an anode and the second electrode 12 is a cathode. Alternatively, the first electrode 11 may be cathode and the second electrode 12 an anode.

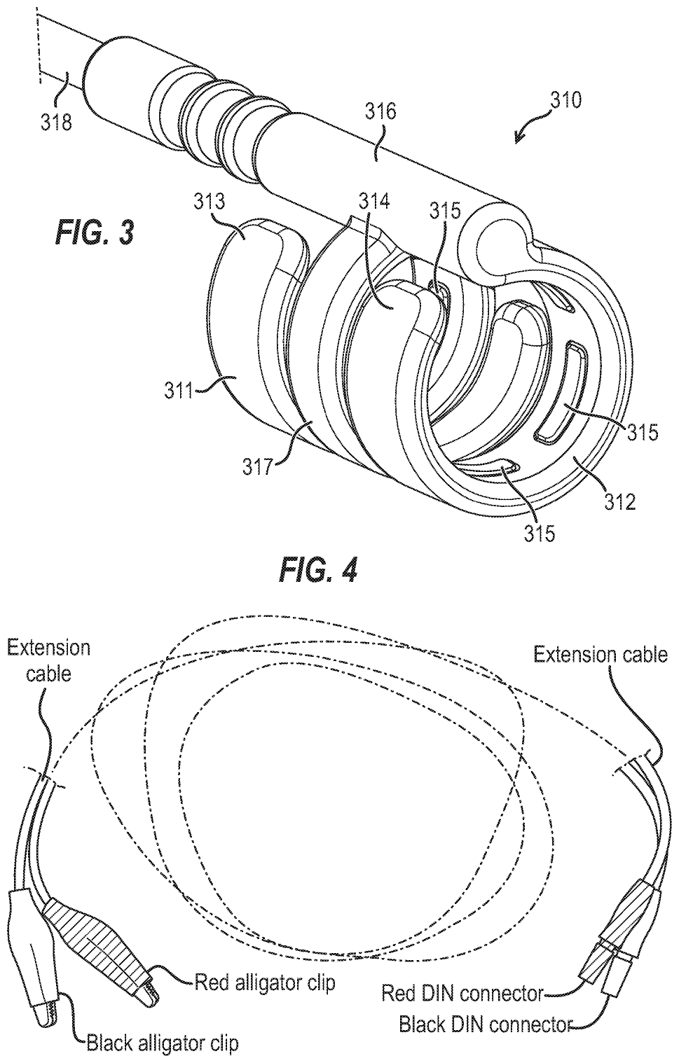

An exemplary FIG. 3 illustrates a schematic presentation of a cuff electrode assembly 310 in accordance with aspects of this invention. The cuff electrode assembly 310 may accommodate the pulsating features of the SpA and may effectively stimulate the SpA NVB in humans. The cuff electrode assembly 310 may be designed to be an implantable lead or neural interface to interface with one or more nerves located around the periphery of the splenic artery. Additionally, the cuff electrode assembly 310 may be and may support the laparoscopic implantation for cuff placement around the splenic NVB. The cuff electrode assembly 310 represents an extravascular bipolar electrode neural interface. The cuff electrode assembly 310 may include a flexible structure. The cuff electrode assembly 310 may include two arms at either end of the device, such as electrode arms 311, 312, which may have open ends 313, 314, respectively. The electrode arms 311, 312 may each be in a C-ring configuration and contain one or more electrodes and/or electrode arrays, such as arrays 315. A center arm portion 317 may be affixed to a spinal portion 316, as are the closed ends of electrode arms 311, 312. The spinal portion 316 may provide mechanical and electrical connection between the electrode arms 311, 312. The center arm portion 317 may be utilized for retention on the artery. The center arm portion 317 may not include any electrodes, serving just to retain the neural interface once positioned, but embodiments may include electrodes.

The electrodes and/or arrays 315 may be connected in series via microcoil interconnects, which are in turn serially connected to a conductor or extension cable 318. The conductors 318 may be covered with the same flexible substrate material used to cover the electrode arms 311, 312 and central arm portion 317 over the length of the spinal portion 316 and extending for a short distance from the cuff electrode assembly 310. Before the conductors 318 exit the material of the spinal portion 316 they may also be covered with a silicon lead body tubing to form the lead body conductor.

FIG. 4 exemplifies an embodiment in which the extension cable 318 may be connected to the cuff electrode assembly 310 via the connector, such as protected alligator or crocodile clips 319. Other connectors may be utilized to connect the extension cable 318 to the cuff electrode assembly 310.

In other embodiments, the at least one electrode may comprise a first electrode, a second electrode, and a third electrode, referred to herein as a tripolar electrode configuration. Other embodiments may include multi-polar electrode configurations, with more than three electrodes.

As with the tripolar configuration, the first, second and third electrodes may be positioned along the longitudinal axis of the nerve, and in one example the second electrode may be positioned between the first electrode and the third electrode.

The electrodes may be at least in part insulated from one another by a non-conductive biocompatible material. To this end, a neural interface may comprise a non-conductive biocompatible material which is spaced transversely along the nerve when the device is in use.

Preferred electrode sizes for applying an electrical signal to at least one splenic arterial nerve may include total surface area. The total surface area of the electrodes may be 0.05-10 cm². The total surface area of the electrodes may be 0.05-0.3 cm². Preferably the total surface area of the electrodes is less than 0.3 cm².

In preferred electrode configurations, the width of each of the first electrode 11 and the second electrode 12 may be between 0.1 mm and 10 mm. In one embodiment of electrode configurations, the width of each of the first electrode 11 and the second electrode 12 may be between 0.1 mm and 4 mm. For example, the width may be between 0.1 mm and 2 mm, or between 1 mm and 3 mm, or between 2 mm and 4 mm, or between 2 mm and 3 mm.

### Controller

Referring to FIG. 1, the system of the invention 50 which may comprise a neural interface, may also comprise at least one controller, for example microprocessor 60, which is electrically coupled to the at least one electrode of the neural interface 10 and configured to control the operation of the least one electrode. The at least one controller may be responsible for triggering the beginning and/or end of the signals delivered to the nerve by the at least one electrode. Optionally, the at least one controller may also be responsible for generating and/or controlling the signal parameters.

The at least one controller may be configured to operate in an open-loop fashion, wherein a predefined signal (as described above) is delivered to the nerve in a predefined pattern of application (also as described above) with or without an external trigger, and without any control or feedback mechanism. Alternatively, the at least one controller may be configured to operate in a closed-loop fashion, wherein 30 a signal is applied based on a control or feedback mechanism.

The at least one controller may be constructed so as to generate, in use, a preconfigured and/or operator-selectable signal that is independent of any input in the system 50. The preconfigured and/or operator-selectable signal may be any one of the electrical signals previously described. The signal can be selected by an operator during surgical application. The signal can be added during the course of surgery. The signal can be based on physiological parameters identified pre-surgically or during the course of surgery. In other embodiments, the at least one controller is responsive to an external signal, favorably information (e.g. data) pertaining to one or more physiological parameters of the subject, but still within the confines of the signals previously described.

The at least one controller may be a microprocessor 60 in the system 50, suitable to be implanted in the subject.

Alternatively or additionally, the at least one controller may be a controller external to the subject.

The at least one controller may be triggered upon receipt of a signal generated by an operator, such as a physician or the subject in which the device 106 may be implanted. The at least one controller may also be external to the patient. To that end, the system 50 may additionally comprise an external system 80 comprising a controller 101. An example of such a system is described below with reference to FIG. 2.

External system 80 of wider system 100 is external the system 50 and external to the subject, and comprises controller 101. Controller 101 may be used for controlling and/or externally powering system 50. To this end, controller 101 may comprise a powering unit 102 and/or a programming unit 103. The external system 80 may further comprise a power transmission antenna 104 and a data transmission antenna 105, as further described below.

The least one controller, including microprocessor 60 and controller 101, may be a processor connected to a memory (i.e. a non-transitory computer readable storage medium) carrying an executable computer program comprising code portions which, when loaded and run on the processor, cause the processor to at least control operation of the at least one electrode. By control the operation is it meant that the at least one controller causes the at least one electrode to apply an electrical signal to the nerve using any of the signal parameters and patterns of application previously described.

### Neural Stimulation System

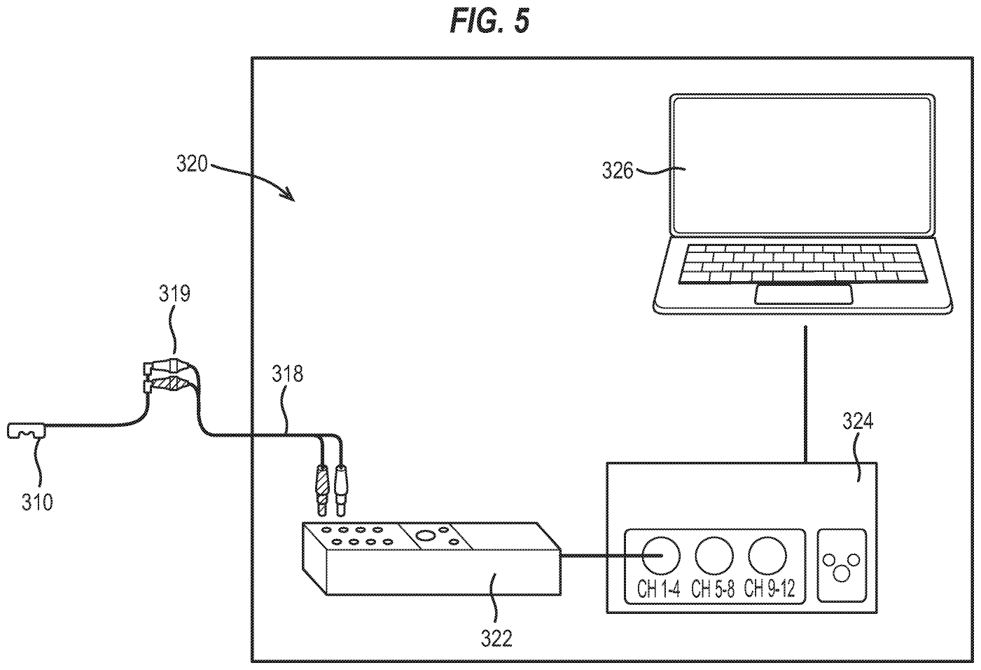

In addition to the neural interface 10 and the at least one controller 60, the system 50 may comprise a signal generator 113 which is configured to deliver the electrical signal described above to the at least one electrode in response to a control operation from the at least one controller. The signal generator may comprise at least one current or voltage source. In one embodiment, the signal generator 113 and associated components may be implanted below the skin. In another embodiment, as illustrated in FIG. 5, the signal generator 113 and the associated components may be external to the patient.

As illustrated in FIG. 5, the proximal end of the lead 310 may contain a connector for the extension cable 318 (for example, model F783ODIN, Fiab SpA, Vicchio, Italy). The extension cable 318 may be connected to the lead 310 via protected crocodile clips 319. On the other end, the extension cable 318 may be connected to an External Pulse Generator system 320 (for example, Inomed Medizintechnik, GmbH, Emmedingen, Germany) or equivalent. The external pulse generator system 320 may consist of an external pulse generator (EPG) 324 and a stimulation adapter 322 connected to a notebook computer 326. The EPG 324 may be, for example, an Inomed ISIS-HC Neurostimulator (model 504185)) or equivalent external pulse generator. The stimulation adapter 322 may be, for example an Inomed Stimulation Adapter 322 (model 540501) (model 504185) or equivalent stimulation adapter. The notebook computer 326 may include pre-installed ISIS Neurostimulator Software or equivalent.

The signal generator 113 may be electrically coupled to the at least one controller and to the at least one electrode. In some embodiments, at least one electrode may be coupled to the signal generator 113 via electrical leads 107. In some embodiments, the electrical leads may be coupled to the interconnectors previously described. Alternatively, the signal generator 113 may be directly integrated with the at least one electrode without leads. In any case, the system 50 may comprise a device 106 and which may comprise DC current blocking output circuits (or AC current blocking output circuits), optionally based on capacitors and/or inductors, on all output channels (e.g. outputs to the at least one electrode, or physiological sensor 111). Additionally, in another embodiment, an external electric pulse generator (EPG) may be utilized for the signal generator 113. The external pulse generator can be coupled to the lead via adapter 322 on a temporary basis during the surgery and then removed.

In addition to the neural interface 10, the at least one electrode, the at least one controller, and the signal generator 113, the system 50 may comprise one or more of the following components: transceiver 110; power source 112; memory 114 (otherwise referred to as a non-transitory computer-readable storage device); physiological sensor 111; and physiological data processing module 115. The physiological sensor 111 and physiological data processing module 115 are referred to herein as a detector. In one embodiment, the transceiver 110; the power source 112; the memory 114; the physiological sensor 111; and the physiological data processing module 115 may be implanted below the skin. In another embodiment, the transceiver 110; the power source 112; the memory 114; the physiological sensor 111; and the physiological data processing module 115 may be external to the patient. The external pulse generator (EPG) 324 can be interfaced to a laptop 326 via a connection cable (for example, a USB cable) to control the operation of the external pulse generator system 320 and the different stimulation parameters/process, and/or record data.

The various components of the system 50 may be part of a single physical device, either sharing a common housing or being a physically separated collection of interconnected components connected by electrical leads, as shown in FIG. 2 and FIG. 5. As an alternative, however, the invention may use a system in which the components are physically separate, and communicate via wires or wirelessly: i.e., intra-operatively the wires can connect through laparoscopic incisions to an external pulse generator. The at least one electrode and the device (e.g. device 106 and/or pulse generating device/pulse generator/signal generator or similar) can be part of a unitary 10 device, or together may form a system (e.g. system 50). In both cases, further components may also be present to form a wider system (e.g. system 100).

For example, in some embodiments, one or more of the following components may be contained in the device 106; power source 112; memory 114; and a physiological data processing module 115. The physiological data processing module 115 in an external system may be coupled separately, for example, the blood flow probe or the monitoring of cardiovascular parameters.

The power source 112 may comprise a current source and/or a voltage source for providing the power for the signal generator 113. The power source 112 may also provide power for the other components of the device 106 and/or system 50, such as the microprocessor 60, memory 114, and implantable transceiver 110. The power source 112 may comprise a battery, the battery may be rechargeable.

Memory 114 may store power data and data pertaining to the one or more physiological parameters. In an external system, the data may be stored in the external pulse generator system 320, the external pulse generator 324, and/or the laptop 326. For instance, memory 114, the external pulse generator system 320, the external pulse generator 324, and/or the laptop 326 may store data pertaining to one or more signals indicative of the one or more physiological parameters detected by detector (e.g. via physiological sensor 111, and/or the one or more corresponding physiological parameters determined via physiological data processing module 115). In addition or alternatively, memory 114, the external pulse generator system 320, the external pulse generator 324, and/or the laptop 326 may store power data and data pertaining to the one or more physiological parameters from external system 80 via the implantable transceiver 110. To this end, the implantable transceiver 110 may form part of a communication subsystem of the wider system 100, as is further discussed below.

Physiological data processing module 115 is configured to process one or more signals indicative of one or more physiological parameters detected by the physiological sensor 111, to determine one or more corresponding physiological parameters. Physiological data processing module 115 may be configured for reducing the size of the data pertaining to the one or more physiological parameters for storing in memory 114 and/or for transmitting to the external system via implantable transceiver 110. Implantable transceiver 110 may comprise one or more antenna (e). The implantable transceiver 110 may use any suitable signaling process such as RF, wireless, infrared and so on, for transmitting signals outside of the body, for instance to wider system 100 of which the system 50 is one part.

The physiological data processing module 115 and the at least one physiological sensor 111 may form a physiological sensor subsystem, also known herein as a detector, either as part of the system 50, part of the device 106, or external to the system.

There may be at least one detector configured to detect one or more physiological parameters. Physiological parameters include inflammatory parameters, e.g. inflammatory markers relating to the reducing of post-surgical complications, such as reduced post-surgical complication rates and days in hospital. For example, reduced post-surgical inflammatory markers may include CRP and/or IL-6, as is further discussed below.

There may be at least one detector configured to detect other physiological parameters such as blood flow rate in the spleen, blood flow rate in the splenic artery, blood flow rate in the splenic vein, spleen volume, neural activity in at least one splenic arterial nerve, or impedance of the at least one electrode.

For example, the detector may be configured for detecting blood flow using intra- or peri-vascular flow tubes in or around the artery or vein. Alternatively, the detector may detect splenic artery contraction and blood flow changes using electrical impedance tomography, electrical impedance, stimulator voltage compliance, Doppler flow, splenic tissue perfusion, ultrasound, strain measurement, or pressure.

In other examples, the detector may be configured for detecting neural activity of at least one splenic arterial nerve using an electrical sensor. When the detector is configured for detecting neural activity of a single splenic arterial nerve, the detector may detect action potentials. When the detector is configured for detecting neural activity of a plurality of splenic arterial nerve, the detector may detect compound action potentials.

In further examples, the detector may be configured for detecting spleen volume using ultrasound.

In other examples, the detector may be configured to detect impedance of the at least one electrode using an impedance meter, favorably a low-current AC (e.g. 1 kHz) impedance meter. In particular, the detector may detect impedance between the at least one electrode and ground, and/or between pairs of electrodes of the at least one electrode (where there is a plurality of electrodes). In such examples, the at least one electrode is suitable for placement on or around the nerve.

The physiological parameters determined by the detector may be used to trigger the microprocessor 60 to deliver a signal of the kinds described above to the nerve using the at least one electrode. Upon receipt of the signal indicative of a physiological parameter received from physiological sensor 111, the physiological data processor 115 may determine the physiological parameter of the subject, and the evolution of the disease, by calculating in accordance with techniques known in the art. For instance, if a signal indicative of excessive cytokine (e.g. TNF) concentration in the circulation is detected, the processor may trigger delivery of a signal which dampens secretion of the respective signaling molecule, as described elsewhere herein.

In some embodiments, controller 101 may be configured to make adjustments to the operation of the system 50. For instance, it may transmit, via a communication subsystem (discussed further below), physiological parameter data pertaining to a normal level of signaling molecules secreted from the spleen. The data may be specific to the subject into which the device is implanted. The controller 101 may also be configured to make adjustments to the operation of the power source 112, signal generator 113 and processing elements 60, 115 and/or electrodes in order to tune the signal delivered to the nerve by the neural interface 10.

As an alternative to, or in addition to, the ability of the system 50 and/or device 106 to respond to physiological parameters of the subject, the microprocessor 60 may be triggered upon receipt of a signal generated by an operator (e.g. a physician or the subject in which the system 50 is implanted). To that end, the system 50 may be part of a wider system 100 which comprises external system 80 and controller 101, as is further described below.

### Beyond the Neural Stimulation System

The neural stimulation system 50 may be part of a wider system 100 that includes a number of subsystems, for example the external system 80, see FIG. 2 and FIG. 5. The external system 80 may be used for powering and programming the neural stimulation system 50 through human skin and underlying tissues. In the embodiment when the neural stimulation system 50 is external to the patient, the external system 80 may still be used for powering and programming the neural stimulation system 50.

The external subsystem 80 may comprise, in addition to controller 101, one or more of: a powering unit 102, for wirelessly recharging the battery of power source 112 used to power the device 106; and, a programming unit 103 configured to communicate with the implantable transceiver 110. The programming unit 103 and the implantable transceiver 110 may form a communication subsystem. In some embodiments, powering unit 102 is housed together with programing unit 103. In other embodiments, they can be housed in separate devices.

The external subsystem 80 may also comprise one or more of: power transmission antenna 104; and data transmission antenna 105. Power transmission antenna 104 may be configured for transmitting an electromagnetic field at a low frequency (e.g., from 30 kHz to 10 MHz). Data transmission antenna 105 may be configured to transmit data for programming or reprogramming the device 106, and may be used in addition to the power transmission antenna 104 for transmitting an electromagnetic field at a high frequency (e.g., from 1 MHz to 10 GHZ). The temperature in the skin will not increase by more than 2 degrees Celsius above the surrounding tissue during the operation of the power transmission antenna 104. The at least one antennae of the implantable transceiver 110 may be configured to receive power from the external electromagnetic field generated by power transmission antenna 104, which may be used to charge the rechargeable battery of power source 112.

The power transmission antenna 104, data transmission antenna 105, and the at least one antennae of implantable transceiver 110 have certain characteristics such a resonant frequency and a quality factor (Q). One implementation of the antenna (e) is a coil of wire with or without a ferrite core forming an inductor with a defined inductance. This inductor may be coupled with a resonating capacitor and a resistive loss to form the resonant circuit. The frequency is set to match that of the electromagnetic field generated by the power transmission antenna 105. A second antenna of the at least one antennae of implantable transceiver 110 can be used in system 50 for data reception and transmission from/to the external system 80. If more than one antenna is used in the system 50, these antennae are rotated 30 degrees from one another to achieve a better degree of power transfer efficiency during slight misalignment with the with power transmission antenna 104.

External system 80 may comprise one or more external body-worn physiological sensors 121 (not shown) to detect signals indicative of one or more physiological parameters. The signals may be transmitted to the system 50 via the at least one antennae of implantable transceiver 110. Alternatively or additionally, the signals may be transmitted to the external system 50 and then to the system 50 via the at least one antennae of implantable transceiver 110. As with signals indicative of one or more physiological parameters detected by the implanted physiological sensor 111, the signals indicative of one or more physiological parameters detected by the external sensor 121 may be processed by the physiological data processing module 115 to determine the one or more physiological parameters and/or stored in memory 114 to operate the system 50 in a closed-loop fashion. The physiological parameters of the subject determined via signals received from the external sensor 121 may be used in addition to alternatively to the physiological parameters determined via signals received from the implanted physiological sensor 111.

For example, in a particular embodiment a detector external to the device may include a non-invasive blood flow monitor, such as an ultrasonic flowmeter and/or a non-invasive blood pressure monitor, and determining changes in physiological parameters, in particular the physiological parameters described above. As explained above, in response to the determination of one or more of these physiological parameters, the detector may trigger delivery of signal to a splenic arterial nerve by the at least one electrode, or may modify the parameters of the signal being delivered or a signal to be delivered to the nerve by the at least one electrode in the future.

The system 100 or the external system 80 may include a safety protection feature that discontinues the electrical stimulation of the nerve in the following exemplary events: abnormal operation of the system 50 (e.g. overvoltage); abnormal readout from an implanted physiological sensor 111 (e.g. temperature increase of more than 2 degrees Celsius or excessively high or low electrical impedance at the electrode-tissue interface); abnormal readout from an external body-worn physiological sensor 121 (not shown); or abnormal response to stimulation detected by an operator (e.g. a physician or the subject). The safety precaution feature may be implemented via controller 101 and communicated to the system 50, or internally within the system 50.

The external system 80 may comprise an actuator 120 (not shown) which, upon being pressed by an operator (e.g. a physician or the subject), will deliver a signal, via controller 101 and the respective communication subsystem, to trigger the microprocessor 60 of the system 50 to deliver a signal to the nerve by the at least one electrode.

The external system 80 may comprise a display 109 for the microcontroller 60 or the controller 101 to alert the operator (e.g. a physician or the subject) to a state of the system or of the subject. The display 109 may be a monitor such as an LED monitor, or may be a visual indicator such as an LED.

System 100, including the external system 80, but in particular system 50, is preferably made from, or coated with, a biostable and biocompatible material. This means that the system is both protected from damage due to exposure to the body's tissues and also minimizes the risk that the system elicits an unfavorable reaction by the host (which could ultimately lead to rejection). The material used to make or coat the system should ideally resist the formation of biofilms. Suitable materials include, but are not limited to, poly(p-xylylene) polymers (known as Parylenes) and polytetrafluoroethylene. The device 106 of the invention may generally weigh less than 50 g.

The invention also provides a method of reversibly stimulating neural activity in the spleen and/or in one or more nerves supplying the spleen for reducing post-operative surgical complications. The method comprises the following steps:
A. providing a system of the invention;
B. positioning an electrode and/or a neural interface in signaling contact with the spleen and/or one or more nerves; and
C. controlling the operation of the least one electrode/stimulator with at least one controller to apply an electrical signal and/or stimulation to the nerve.

Referring to step A of the method, the system of the invention provided may comprise a transducer. The transducer may be a neural interface with at least one electrode, and at least one controller. The transducer may also include an ultrasonic signal. Any other feature of the system described herein may also be provided.

In step B, the transducer is favorably positioned in signaling contact with the spleen and/or the one or more nerves. In step B, the transducer is favorably positioned in physical contact with the spleen and/or the one or more nerves.

In step C, the at least one controller controls the operation of the at least one transducer to apply the electrical signal or ultrasonic signal to the spleen and/or the one or more nerves.

The invention also provides a method of reversibly stimulating neural activity in the spleen and/or one or more nerves supplying the spleen for reducing post-operative surgical complications, wherein the nerve is associated with a neurovascular bundle (e.g. a splenic arterial nerve). The method comprises the following steps:
A. providing a system of the invention;
B. positioning an electrode and/or a neural interface in signaling contact with one or more nerves; and
C. controlling the operation of the least one electrode/stimulator with at least one controller to apply an electrical signal and/or stimulation to the nerve.

Referring to step A of the method, the system of the invention provided may comprise a neural interface with at least one electrode, and at least one controller. Any other feature of the system described herein may also be provided.

In step B, the electrode and/or neural interface is favorably positioned in signaling contact with the spleen and/or the one or more nerves. In a further favorable embodiment, the electrode is in physical contact with the spleen and/or one or more nerves. In an additional embodiment, the electrode and neural interface are in physical contact with the one or more nerves.

In step C, the at least one controller controls the operation of the at least one electrode to apply the electrical signal to the nerve. The electrical signal may be similar to an electrical signal used to stimulate neural activity described above in that it may have the same waveform, and also apply the same charge density per phase to the nerve. In some embodiments, the overall charge applied to the nerve is higher. This can be achieved by using continuous signal application instead of periodic signal application, and/or by using a higher frequency than the frequencies described for continuous signal application above.

The invention also provides another method of reversibly stimulating neural activity in the spleen and/or one or more nerves supplying the spleen for reducing post-operative surgical complications. The method comprises the following steps:
A. providing a system of the invention;
B. positioning at least one transducer in signaling contact with the spleen and/or one or more nerves; and
C. controlling the operation of the least one transducer/stimulator with at least one controller to apply a signal and/or stimulation to the nerve.

Referring to step A of the method, the system of the invention provided may comprise a transducer. The transducer may be a neural interface with at least one electrode, and at least one controller. The transducer may also include an ultrasonic signal. Any other feature of the system described herein may also be provided.

In step B, the transducer is favorably positioned in signaling contact with the spleen and/or the one or more nerves. In step B, the transducer is favorably positioned in physical contact with the spleen and/or the one or more nerves.

In step C, the at least one controller controls the operation of the at least one transducer to apply the electrical signal or ultrasonic signal to the spleen and/or the one or more nerves.

The invention also provides another method of reversibly stimulating neural activity in the spleen and/or one or more nerves supplying the spleen for reducing post-operative surgical complications, wherein the nerve is associated with a neurovascular bundle (e.g. a splenic arterial nerve). The method comprises the following steps:
A. providing a system of the invention;
B. positioning at least one transducer in signaling contact with the spleen and/or one or more nerves; and
C. controlling the operation of the least one transducer/stimulator with at least one controller to apply a signal and/or stimulation to the nerve.

Referring to step A of the method, the system of the invention provided may comprise a transducer. The transducer may be a neural interface with at least one electrode, and at least one controller. The transducer may also include an ultrasonic signal. Any other feature of the system described herein may also be provided.

In step B, the transducer is favorably positioned in signaling contact with the spleen and/or the one or more nerves. In step B, the transducer is favorably positioned in physical contact with the spleen and/or the one or more nerves.

In step C, the at least one controller controls the operation of the at least one transducer to apply the electrical signal or ultrasonic signal to the spleen and/or the one or more nerves.

### Post-Surgical Complications

The invention is useful for reducing and treating post-surgical complications. Post-surgical complications may include, but not be limited to one or more of the following: pneumonia, anastomotic leakage, and postoperative ileus. Cardiac complications include: cardiac arrest, cardiac ischemia/infarction, pericarditis, congestive heart failure and a-/dysrhythmias requiring intervention. Pulmonary complications include: (aspiration) pneumonia, pleural effusion/empyema, pneumothorax and atelectasis requiring intervention and acute respiratory distress syndrome and respiratory insufficiency requiring prolonged treatment or reintubation.

The invention is useful for reducing post-surgical complications related to shock. Shock is a severe drop in blood pressure that causes a dangerous reduction of blood flow throughout the body. Shock may be caused by blood loss, infection, brain injury, or metabolic problems. Treatment for shock may include any or all of the following: stopping any blood loss; helping with breathing (with mechanical ventilation if needed); reducing heat loss; giving intravenous (IV) fluids or blood; providing oxygen; and/or prescribing medicines, for example, to raise blood pressure.

The invention is useful for reducing post-surgical complications related to hemorrhaging. Hemorrhaging means bleeding. The rapid blood loss from the site of surgery, for example, can lead to shock. Treatment of rapid blood loss may include any or all of the following: IV fluids or blood plasma, blood transfusion, and/or more surgery to control the bleeding.

The invention is useful for reducing post-surgical complications related to wound infection. When bacteria enter the site of surgery, an infection can result. Infections can delay healing. Wound infections can spread to nearby organs or tissue, or to distant areas through the blood stream. Treatment of wound infections may include any or all of the following: antibiotics and/or surgery or procedure to clean or drain the infected area.

The invention is useful for reducing post-surgical complications related to venous thromboembolism (VTE). Deep vein thrombosis (DVT) and pulmonary embolism (PE) together are referred to as VTE. A deep vein thrombosis is a blood clot in a large vein deep inside a leg, arm, or other parts of the body. Symptoms are pain, swelling, and redness in a leg, arm, or other area.

The invention is useful for reducing post-surgical complications related to pulmonary embolism (PE). The clot can separate from the vein and travel to the lungs. This forms a pulmonary embolism. In the lungs, the clot can cut off the flow of blood. This is a medical emergency and may cause death. Symptoms of PE may include chest pain, trouble breathing, coughing (may cough up blood), sweating, fast heartbeat, and fainting. Treatment depends on the location and size of the blood clot. Treatments of PE may include any or all of the following: anticoagulant medicines (blood thinners to prevent further clotting), thrombolytic medicines (to dissolve clots), and/or surgery or other procedures.

The invention is useful for reducing post-surgical complications related to lung (pulmonary) complications. Sometimes, pulmonary complications arise due to lack of deep breathing and coughing exercises within 48 hours of surgery. They may also result from pneumonia or from inhaling food, water, or blood, into the airways. Symptoms may include wheezing, chest pain, fever, and cough (among others).

The invention is useful for reducing post-surgical complications related to reaction to anesthesia. Although rare, allergies to anesthetics do occur. Symptoms can range from mild to severe. Treatment of allergic reactions includes stopping specific medicines that may be causing allergic reactions. Also, administering other medicines to treat the allergy.

### Reducing Post-Operative Surgical Complications

Post-operative surgical complications can be reduced in various ways, but typically involves determining a reduction in one or more inflammatory markers of the subject. A useful inflammatory marker of the invention may be at least CRP plasma levels. Reduced CRP levels correlate with levels predicting a reduction in post-operative complications, e.g. criterion with CRP levels below 215 mg/L, or predicting safe discharge from hospital, criterion with CRP levels below 75 mg/L. Another useful inflammatory marker of the invention to reduce post-operative surgical complications may be decreasing IL-6 levels after surgery.

As used herein, an “improvement in a determined physiological parameter” and/or “reduced post-operative surgical complications” and/or “reduction in inflammatory markers/parameters” is taken to mean that, for any given physiological parameter and/or inflammatory marker, an improvement is a change in the value of that parameter and/or marker in the subject towards the normal value or normal range for that value—i.e. towards the expected value in a healthy subject. For example, the value or range for a subject not experiencing an inflammatory response, i.e. an uninflamed healthy subject. As used herein, “worsening of a determined physiological parameter” is taken to mean that, for any given physiological parameter, worsening is a change in the value of that parameter and/or marker in the subject away from the normal value or normal range for that value—i.e. away from the expected value in a healthy subject, for example, away from the expected value or range for an uninflamed healthy subject.

For the purpose of the metrics described herein, it will be understood that a decrease or increase is a nonzero positive value (e.g. 1%, 2%, 3%, or 4%).

By stimulating the splenic arterial nerve, the level of CRP levels on various post-operative days, may decrease, for example, by: ≤5%, ≤10%, ≤15%, ≤20%, ≤25%, ≤30%, ≤35%, ≤40%, ≤45%, ≤50%, ≤60%, ≤70%, ≤80%, ≤90%, ≤95%, or ≤100%. For example, the decrease may be ≥5%, ≥10%, ≥15%, ≥20%, ≥25%, ≥30%, ≥35%, ≥40%, ≥45%, ≥50%, ≥60%, ≥70%, ≥80%, ≥90%, or ≥95%. Any combination of the upper and lower limits above is also possible.

Improvement in a determined inflammatory marker according to the invention to improve post-operative surgical complications is indicated by one or more of the group consisting of: reduced CRP levels and/or decreasing IL-6 levels after surgery. Other physiological parameters may indicate to improvement for post-operative surgical complications from one or more of the group consisting of: a reduction in a pro-inflammatory cytokine, an increase in an anti-inflammatory cytokine and/or resolving mediator, an increase in a catecholamine, the level of erythrocytes, a change in an immune cell population, a change in an immune cell surface co-stimulatory molecule, a reduction or increase in a factor involved in the inflammation cascade, and a change in the level of an immune response mediator. The invention might not lead to a change in all of these parameters.

By stimulating a splenic nerve, the spleen may: (a) decrease the secretion of a pro-inflammatory cytokine compared to baseline secretion; and/or (b) increase the secretion of an anti-inflammatory cytokines and/or resolving mediators compared to baseline secretion. For example, the decrease in a pro-inflammatory cytokine secretion may be by: ≤5%, ≤10%, ≤15%, ≤20%, ≤25%, ≤30%, ≤35%, ≤40%, ≤45%, ≤50%, ≤60%, ≤70%, ≤80%, ≤90% or ≤95%. In another example, the decrease in a pro-inflammatory cytokine secretion may be by: ≥5%, ≥10%, ≥15%, ≥20%, ≥25%, ≥30%, ≥35%, ≥40%, ≥45%, ≥50%, ≥60%, ≥70%, ≥80%, ≥90% or ≥95%. Any combination of the upper and lower limits above is also possible. The increase in an anti-inflammatory cytokine secretion may be by: ≤5%, ≤10%, ≤15%, ≤20%, ≤25%, ≤30%, ≤35%, ≤40%, ≤45%, ≤50%, ≤60%, ≤70%, ≤80%, ≤90%, ≤95%, ≤100%, ≤150%, ≤200%, or ≤500-1000%. The increase in an anti-inflammatory cytokine secretion may be by: ≥5%, ≥10%, ≥15%, ≥20%, ≥25%, ≥30%, ≥35%, ≥40%, ≥45%, ≥50%, ≥60%, ≥70%, ≥80%, ≥90%, ≥95%, ≥100%, ≥150%, ≥200%, or ≥500-1000%. Any combination of the upper and lower limits above is also possible.

Once the cytokine is secreted into the circulation, its concentration in circulation is diluted. Stimulation of the splenic nerve may result in: (a) a decrease in the level of a pro-inflammatory cytokine in the plasma or serum by ≤5%, ≤10%, ≤15%, ≤20%, ≤25%, ≤30%, ≤35%, ≤40%, ≤45%, ≤50%, ≤60%, ≤70%, ≤80%, ≤90%, or ≤95%, or a decrease in the level of a pro-inflammatory cytokine in the plasma or serum by ≥5%, ≥10%, ≥15%, ≥20%, ≥25%, ≥30%, ≥35%, ≥40%, ≥45%, ≥50%, ≥60%, ≥70%, ≥80%, ≥90%, or ≥95%; and/or (b) an increase in the level of an anti-inflammatory cytokine and/or resolving mediator in the plasma or serum by ≤5%, ≤10%, ≤15%, ≤20%, ≤25%, ≤30%, ≤35%, ≤40%, ≤45%, ≤50%, ≤60%, ≤70%, ≤80%, ≤90%, ≤95%, ≤100%, ≤150% or ≤200%, or an increase in the level of an anti-inflammatory cytokine and/or resolving mediator in the plasma or serum by ≥5%, ≥10%, ≥15%, ≥20%, ≥25%, ≥30%, ≥35%, ≥40%, ≥45%, ≥50%, ≥60%, ≥70%, ≥80%, ≥90%, ≥95%, ≥100%, ≥150% or ≥200%.

Any combination of the upper and lower limits above is also possible. Favorably, the level in the serum is measured.

By stimulating the splenic nerve, the level of catecholamine (e.g. norepinephrine or epinephrine), e.g. its level in the spleen or splenic vein, may increase, for example, by: ≤5%, ≤10%, ≤15%, ≤20%, ≤25%, ≤30%, ≤35%, ≤40%, ≤45%, ≤50%, ≤60%, ≤70%, ≤80%, ≤90%, ≤95%, ≤100%, ≤150% or ≤200%. The level of catecholamine (e.g. norepinephrine or epinephrine), e.g. its level in the spleen or splenic vein, may increase, for example, by: ≥5%, ≥10%, ≥15%, ≥20%, ≥25%, ≥30%, ≥35%, ≥40%, ≥45%, ≥50%, ≥60%, ≥70%, ≥80%, ≥90%, ≥95%, ≥100%, ≥150% or ≥200%. Any combination of the upper and lower limits above is also possible. For example, stimulating a splenic arterial nerve can decrease the level of a pro-inflammatory cytokine (e.g. TNFα) in the serum by 30%-70%.

Pro-inflammatory cytokines are known in the art. Examples of these include tumor necrosis factor (TNF; also known as TNFα or cachectin), interleukin (IL)-1α, IL-1β, IL-6, and IL-8. Decreasing these pro-inflammatory cytokines can decrease the progression of an inflammatory condition.

Anti-inflammatory cytokines are known in the art. IL-10 is an anti-inflammatory cytokine. An increase in IL-10 can counteract inflammatory effects.

Methods of assessing these physiological parameters are known in the art. Detection of any of the measurable parameters may be done before, during and/or after modulation of neural activity in the nerve, such as heart rate and systemic blood pressure, and arterial blood flow.

Quantitative changes of the biological molecules (e.g. cytokines and pro-resolving mediators) can be measured in a living body sample such as urine or plasma. Detection of the biological molecules may be performed directly on a sample taken from a subject, or the sample may be treated between being taken from a subject and being analyzed. For example, a blood sample may be treated by adding anticoagulants (e.g. EDTA), followed by removing cells and cellular debris, leaving plasma containing the relevant molecules (e.g. cytokines and pro-resolving mediators) for analysis. Alternatively, a blood sample may be allowed to coagulate, followed by removing cells and various clotting factors, leaving serum containing the relevant molecules (e.g. cytokines and pro-resolving mediators) for analysis.

As used herein, a physiological parameter is not affected by the modulation (e.g. stimulation) of the splenic neural activity if the parameter does not change (in response to nerve modulation) from the normal value or normal range for that value of that parameter exhibited by the subject or subject when no intervention has been performed i.e. it does not depart from the baseline value for that parameter.

Such a physiological parameter may be mean arterial blood pressure, heart rate or glucose metabolism. Suitable methods for determining changes in any these physiological parameters would be appreciated by the skilled person.

The skilled person will appreciate that the baseline for any neural activity or physiological parameter in a subject need not be a fixed or specific value, but rather can fluctuate within a normal range or may be an average value with associated error and confidence intervals. Suitable methods for determining baseline values are well known to the skilled person.

As used herein, a physiological parameter is determined in a subject when the value for that parameter exhibited by the subject at the time of detection is determined. A detector (e.g. a physiological sensor subsystem, a physiological data processing module, a physiological sensor, etc.) is any element able to make such a determination.

Thus, in certain embodiments, the invention further comprises a step of determining one or more physiological parameters of the subject, wherein the signal is applied only when the determined physiological parameter meets or exceeds a predefined threshold value. In such embodiments wherein more than one physiological parameter of the subject is determined, the signal may be applied when any one of the determined physiological parameters meets or exceeds its threshold value, alternatively only when all of the determined physiological parameters meet or exceed their threshold values. In certain embodiments wherein the signal is applied by a system of the invention, the system further comprises at least one detector configured to determine the one or more physiological parameters of the subject.

In certain embodiments, the physiological parameter is an action potential or pattern of action potentials in a nerve of the subject, wherein the action potential or pattern of action potentials is associated with the condition that is to be treated.

It will be appreciated that any two physiological parameters may be determined in parallel embodiments, the controller is coupled detect the pattern of action potentials tolerance in the subject.

A predefined threshold value for a physiological parameter is the minimum (or maximum) value for that parameter that must be exhibited by a subject or subject before the specified intervention is applied. For any given parameter, the threshold value may be defined as a value indicative of a pathological state (e.g. an inflamed condition) or a disease state. The threshold value may be defined as a value indicative of the onset of a pathological state or a disease state. Thus, depending on the predefined threshold value, the invention can be used as a treatment. Alternatively, the threshold value may be defined as a value indicative of a physiological state of the subject (that the subject is, for example, inflamed condition). Appropriate values for any given physiological parameter would be simply determined by the skilled person (for example, with reference to medical standards of practice).

Such a threshold value for a given physiological parameter is exceeded if the value exhibited by the subject exceeds a threshold that in a healthy state, for example, a non-inflamed condition, a non-disease state, or a non-pathological state.

A subject of the invention may, in addition to receiving stimulation, receive medicine. For instance, a subject having receiving neural stimulation according to the invention may receive for example, dexamethasone, weak or strong opioids, and/or nonsteroidal anti-inflammatory drugs (NSAIDS). Thus, the invention provides the use of these medicines, and other medicines as necessary or allowed, in combination with a system of the invention.

### General

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium.

Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously. This acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

Those skilled in the art will realize that storage devices utilized to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a DSP, programmable logic array, or the like.

Unless otherwise indicated each embodiment as described herein may be combined with another embodiment as described herein. The term “comprising” encompasses “including” as well as “consisting” e.g. a composition “comprising” X may consist exclusively of X or may include something additional e.g. X+Y.

It will be understood that the benefits and advantages described above may relate to one embodiment or may relate to several embodiments. The embodiments are not limited to those that solve any or all of the stated problems or those that have any or all of the stated benefits and advantages.

It will be understood that the above description of a preferred embodiment is given by way of example only and that various modifications may be made by those skilled in the art. Although various embodiments have been described above with a certain degree of particularity, or with reference to one or more individual embodiments, those skilled in the art could make numerous alterations to the disclosed embodiments without departing from the scope of this invention.

### EXAMPLES OF SPECIFIC EMBODIMENTS

### Example 1: Electrophysiology in a Porcine Model

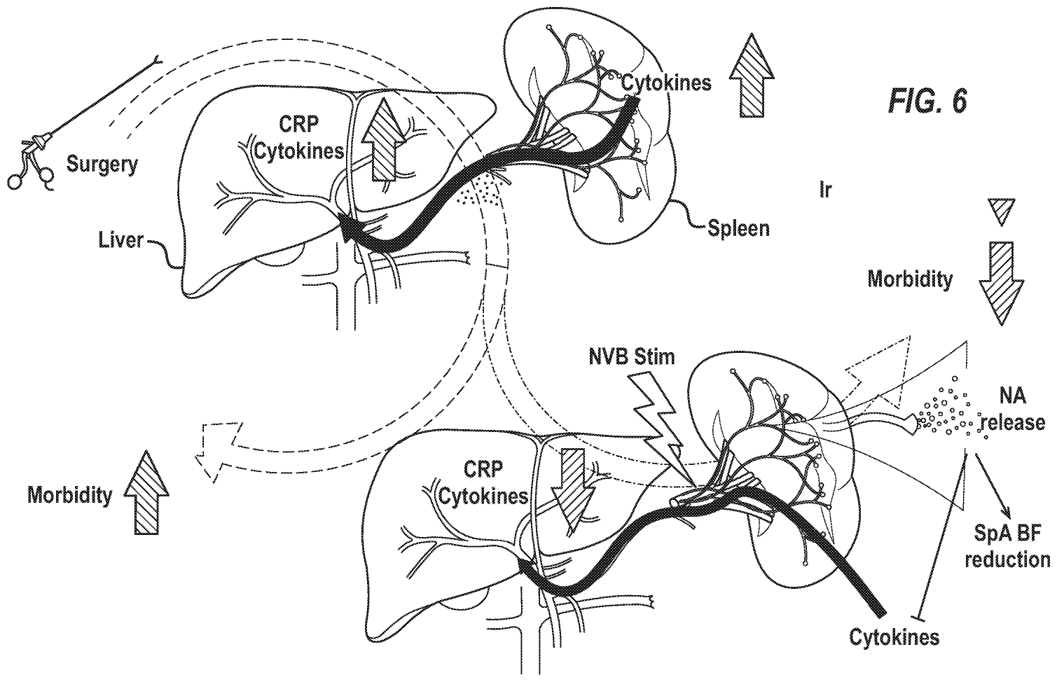

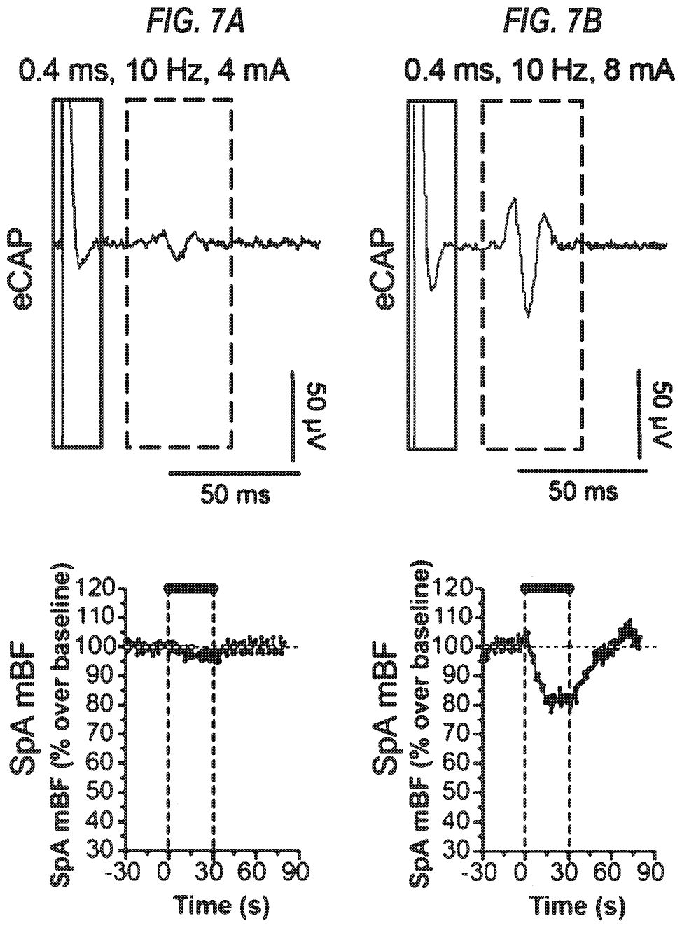

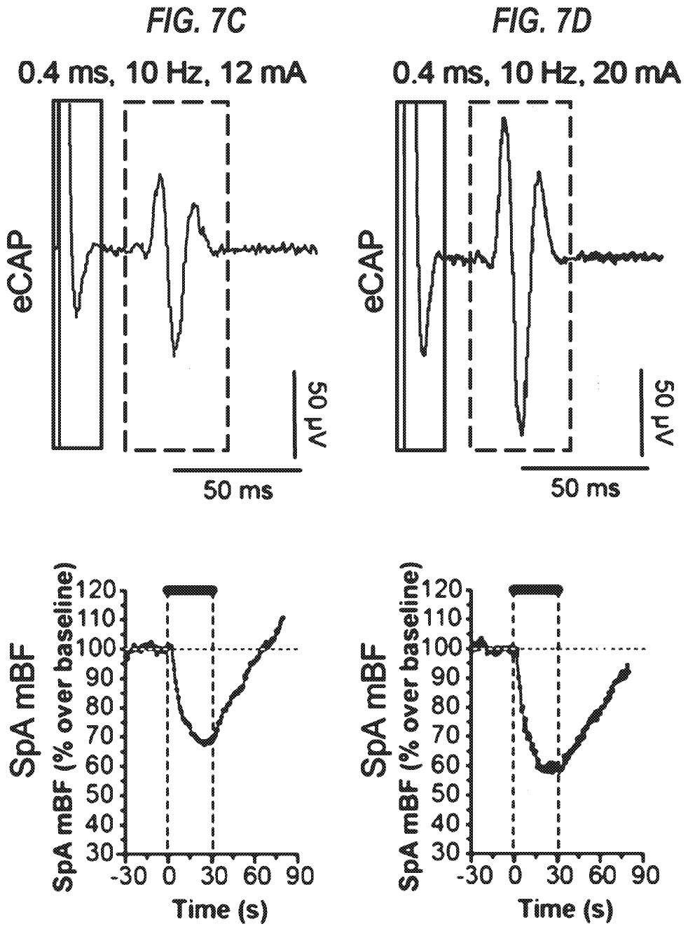

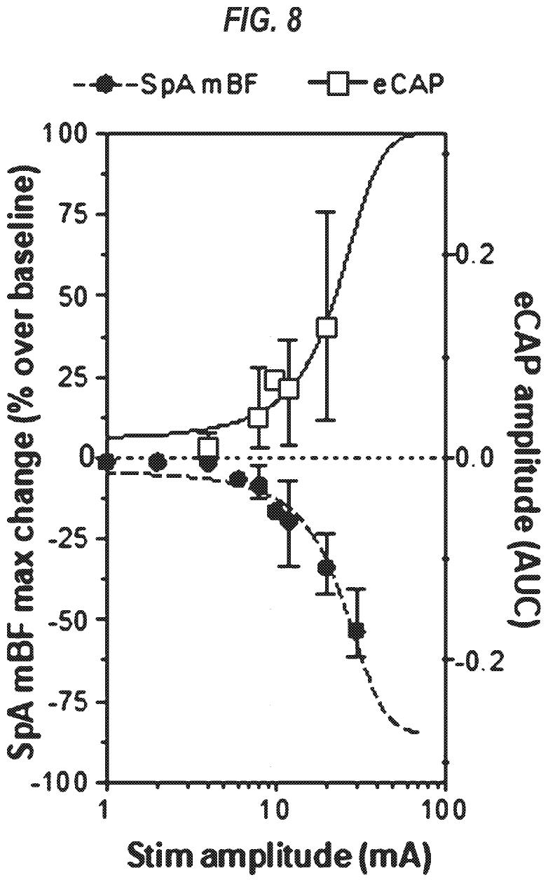

Laparoscopic placement of the cuff and selection of stimulation parameters were performed using acute electrophysiology experiments in terminally anesthetized farm pigs, a relevant model of the human splenic nerve (Donegà PNAS (2018)). The electrodes were implanted around the splenic NVB and multiple stimulations with increasing amplitude were performed while measuring changes in SpA BF and the relative evoked compound action potential (eCAP). Stimulation of the NVB with biphasic, bipolar rectangular pulses at 0.4 ms pulse width (PW, per phase) and 10 Hz caused amplitude-dependent reduction in SpA BF, as illustrated in FIGS. 5 and 6. Importantly the observed changes in SpA BF correlated with the recruitment of SpN axons as shown by the increasing amplitude of the recorded eCAP. This correlation was in line with previous work performed using pre-clinical commercially available electrodes (Donega PNAS (2021)). In the farm pigs a transit time flow (TTF) probe that encircles a portion of the artery was used to detect the BF change.

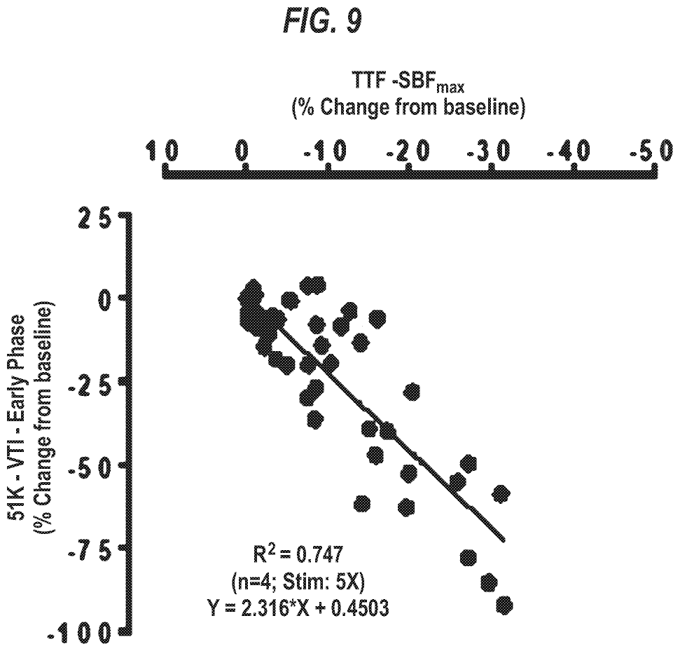

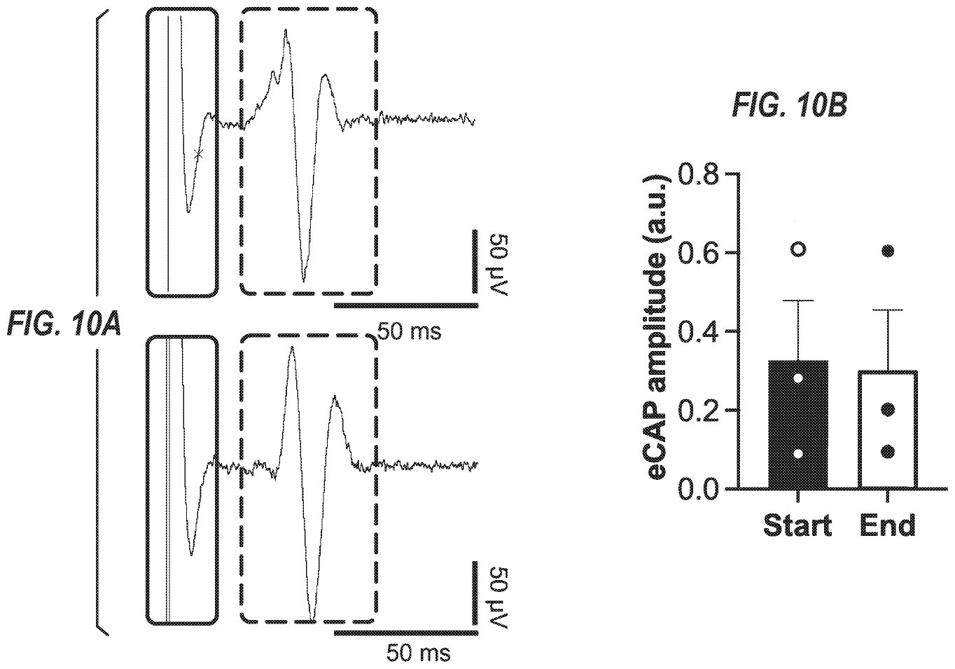

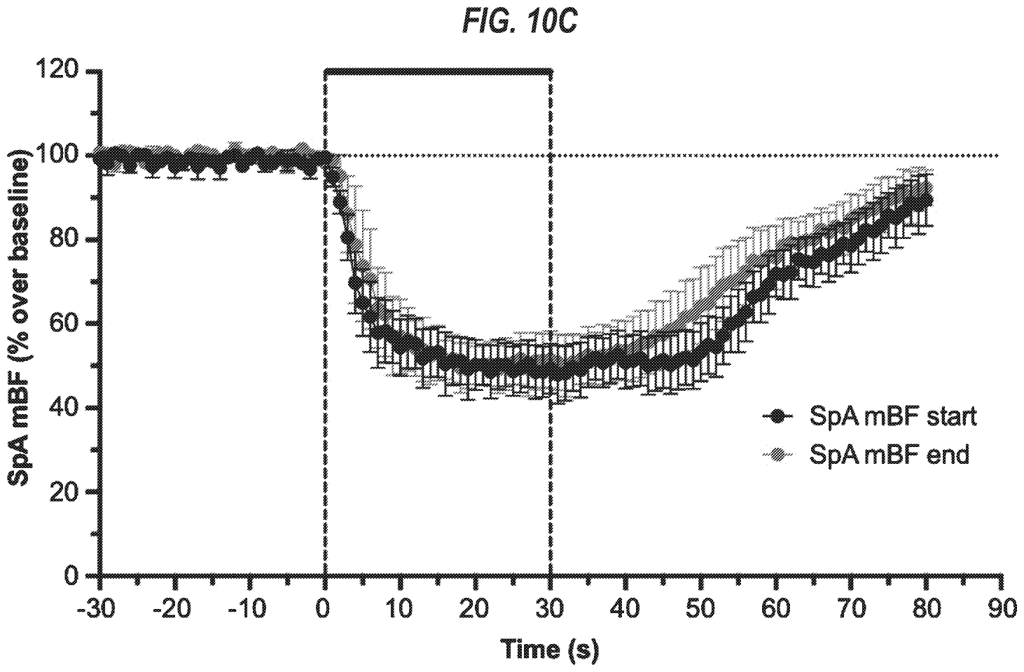

Another pig study was performed to validate the use of SpA BF as biomarker of nerve engagement when using a Doppler ultrasound system compatible to clinical use. The stimulation induced changes in and Velocity Time Integral (VTI) and Peak Systolic Velocity (PSV), measured using the ultrasound transducers was significantly (p<0.0001) and strongly correlated (R2=0.75-0.77) with the changes simultaneously measured using the TTF probe, as illustrated in FIG. 9. Importantly, acute delivery of >50 consecutive stimulations did not affect the activation profile of the SpN in pig, as illustrated in FIGS. 10A, 10B, and 10C. To further demonstrate the safety of the investigational procedure a GLP study accessing the safety of laparoscopically implanting and removing electrodes around the NVB and deliver stimulations while measuring SpA BF using the Doppler flow probe was performed in pigs. The study showed that there were no nerve pathology differences caused by placement, stimulation and removal of the cuff and placement of the ultrasound probe, between the surgical sham and the treated animals. There were no histological effects of the splenic artery or splenic vein observed in any of the animals. In addition, there were no downstream effects on the spleen or pancreas.

All animal studies were ethically reviewed and carried out in accordance with Animals (Scientific Procedures) Act 1986. The protocol was approved by the Royal Veterinary College Animal Welfare and Ethical Review Board and the Galvani Bioelectronics Animal and Scientific Review Committee. All animals were transported and housed under conditions specified in the United Kingdom Animal Welfare Act 2006 and The Welfare of Farm Animals (England) Regulations 2007.

Three farm pigs (female, Large white/British landrace cross; BW 60-70 kg, age 12-14 weeks) were sourced from a commercial pig farm and acclimatized at the research facility for a minimum of 7 days prior to the experiment. Animals were group housed on straw bedding and given food and water ad libitum until 12 hours prior to the experiment, at which point food was withheld. On the day of the experiment, animals were pre-medicated with ketamine (20 mg/Kg) and midazolam (0.5 mg/Kg) administered by intramuscular injection. Fifteen minutes after premedication, a 20 G intravenous catheter was placed in the auricular vein. General anesthesia was induced with propofol (2 mg/kg) administered intravenously. Animals were intubated with an endotracheal tube, and anesthesia was maintained with sevoflurane vaporized in a 50:50 mixture of oxygen and medical air. Following induction of stable anesthesia, bilateral indwelling jugular vein catheters and one femoral arterial catheter were placed under ultrasonographic guidance. Volume-controlled mechanical ventilation was maintained for surgery and instrumentation. Once instrumentation was complete animals were kept on spontaneous ventilation and data collection commenced when animals were in a steady-state as assessed by stable cardiovascular parameters. Routine anesthesia monitoring included vital parameters such as electrocardiogram and invasive arterial blood pressure (systolic, diastolic and mean), central venous pressure; end-tidal CO2 (ETCO2), fraction of inspired oxygen (FiO2), end-tidal sevoflurane (ETSev), pulse oximetry and core body temperature (via rectal probe). Animals were placed in right lateral recumbency, and the left lateral abdomen was clipped and aseptically prepared and draped in a routine fashion. Using aseptic technique, a 20 cm laparotomy incision was made in the second to last intercostal space, and access to the splenic base with associated neurovasculature was aided by the use of rib retractors. For the electrophysiological experiment, the splenic NVB was instrumented as detailed below. At the end of all experiments, animals were humanely euthanized with an overdose of pentobarbital administered intravenously, after which the splenic neurovasculature was rapidly harvested and fixed in 10% neutral buffered formalin (NBF; VWR) for histological analysis.

Surgical procedure was as previously described. In brief, commencing at the origin of the SpA, a 10 mm long segment of the artery with an intact periarterial SpN plexus was carefully separated from the SpV and surrounding loose connective tissue. This splenic NVB was subsequently instrumented with a clinically designed bipolar circumferential cuff electrode (5 mm diameter, Galvani Bioelectronics). Distal (ca 3-5 cm) to this site a minimum of one discrete SpN fascicle was carefully isolated and subsequently instrumented with one cuff electrode (diameter 0.5 mm, length 5.5 mm; cathode surface area: 0.0063-0.01 cm2; #1041.2115.01 or #1041.2112.01; CorTec GmbH) to record eCAP. For monitoring blood flow changes during SpN stimulation, an ultrasonic transit time flow probe (Transonic Systems Inc., USA) was placed around the SpA immediately proximal to the branching of the left gastroepiploic artery from the SpA. Flow changes were continuously monitored via a TS420 perivascular flow module (Transonic Systems Inc.), and measurements were digitally recorded using a 16 channel PowerLab acquisition system (ADInstruments) with LabChart 8 software at 2 kHz sampling frequency. Other parameters, including arterial blood pressure, central venous pressure, ECG, ETCO2, ETSev) were also digitally recorded using a 16 channel PowerLab acquisition system (ADInstruments) with LabChart 8 software at 2 kHz sampling frequency.

Stimulation of the SpN plexus with various intensities, pulse durations and frequencies were performed while eCAP as well as physiological responses were recorded continuously. To ascertain stimulation dose-response effects, the splenic NVB was stimulated via the periarterial cuff electrode using bipolar or monopolar, biphasic or monophasic pulses delivered at 10 Hz and 0.4 ms PW (per phase) for 30 seconds. For cuff performance, some stimulations were delivered at 1 Hz, and sometimes the PW changed (between 0.1-1 ms) as detailed in the result section.

Evoked CAPs were recorded using the small cuffs placed around SpN fascicles dissected off the SpA distally to the stimulation cuff. The signal was amplified and filtered (100-1000 Hz) using a bioamplifier (1800 2-channel microelectrode AC amplifier, A-M System) and a notch-filter (50 Hz). Additionally, when the isolated SpN fascicle was instrumented with more than one cuff electrode, eCAPs were recorded through one cuff electrode while stimulation was applied through the other (this was typically used to assess nerve functionality). Nerve activity was monitored using an oscilloscope, and digitally recorded with a 16-channel PowerLab acquisition system (AD Instruments) and Labchart software with the sampling rate set at 20 kHz.

For monitoring blood flow changes during SpN stimulation, an ultrasonic transit time flow probe (Transonic) was placed around the SpA immediately proximal to the branching of the gastroepiploic artery from the SpA. Flow changes were continuously monitored via a TS420 perivascular flow module (Transonic), and measurements were digitally recorded using a 16 channel PowerLab acquisition system (AD Instruments) with LabChart 8 software at 2 kHz sampling frequency.

Evoked CAPs were averaged (8 pulses) and peak to peak measures performed or signal was rectified for quantification of area under the curve (AUC) of the averaged response. In some cases, when analyzing the eCAP response within the first 10-20 pulses of stimulation, eCAP were not averaged. The relative amplitude or latency (and conduction velocity) of the eCAP were then expressed as % over the first response. When generating a dose-response curve the eCAP values were expressed as % over the response achieved at maximum stimulation intensity used (in this study 30 mA). The conduction velocity of the eCAP components was calculated from the measured distance between the stimulation site and the recording site and the latency of the eCAP signal (measured from the start of the stimulation artefact to the peak or trough of the eCAP response).

Physiology parameters, such as SpA mBF and sMABP, were extracted from LabChart files, down-sampled to obtain 1 value per second from −30 seconds prior to stimulation onset to +90 seconds. Baseline values were then calculated by averaging the 30 s prior to stimulation onset. Each data point was then expressed as % change over the average baseline. When plotting the changes in SpA mBF and sMABP versus the eCAP amplitude, the minimum % value of SpA mBF (between 0 and 90 s) was used.

### Example 2: Intra-Operative Splenic Nerve Stimulation in Human Patients

A single center open label phase 1b trial was conducted to study SpA NVB stimulation intra-operatively in patients undergoing MIE-IL. In these patients the splenic artery is freed during the lymphadenectomy that is performed as part of MIE-IL procedure. This provided the opportunity to confirm feasibility and safety of SpA NVB stimulation with only minor risk of additional surgical trauma. All patients undergoing MIE-IL have a postoperative systemic inflammatory response that can be measured. Studies suggest that systemic inflammation after surgery has a negative impact on surgical outcomes in patients undergoing elective surgery (Fransen L F, J Thorac Dis 2019).

As illustrated in FIGS. 3 and 5, a neural interface 310 was designed to interface the nerves surrounding the splenic artery, as described above and incorporated by reference as detailed above. In this embodiment of a neural interface 310, the distal end of the neural interface 310 may be composed of electrically active electrode arms 311, 312 containing the stimulation electrodes 315, an electrically inert middle arm 317 used for retention on the artery, and a spine 316 which provide mechanical and electrical connection between the arms. As illustrated in FIG. 5, the proximal end of the neural interface 310 contains a connector for the extension cable 318 (for example, model F783ODIN, Fiab SpA, Vicchio, Italy). The extension cable 318 is connected to the neural interface 310 via protected crocodile clips 319. On the other end, the extension cable 318 is connected to an External Pulse Generator system 320 (for example, Inomed Medizintechnik, GmbH, Emmedingen, Germany) or equivalent. The external pulse generator system 320 consists of an external pulse generator (EPG) 324 and a stimulation adapter 322 connected to a notebook computer 326. The EPG 324 may be, for example, an Inomed ISIS-HC Neurostimulator (model 504185)) or equivalent external pulse generator. The stimulation adapter 322 may be, for example an Inomed Stimulation Adapter 322 (model 540501) (model 504185) or equivalent stimulation adapter. The notebook computer 326 may include pre-installed ISIS Neurostimulator Software or equivalent.

The human study demonstrated the safety of applying a stimulation lead, stimulating the NVB and removing the lead in an intra-operative setting. This was assessed by the proportion of participants in whom the lead was successfully applied and removed. Application of a selected cuff is considered successful upon observation that all three arms encircle the NVB. Cuff removal was successful with withdrawal of the lead in its entirety from the abdominal cavity. Furthermore, potential adverse effects from cuff placement and NVB stimulation were evaluated during a seven-day follow-up period.

Splenic artery blood flow changes as measured by the Peak Systolic Velocity (PSV), End Diastolic Velocity (EDV), and the Velocity Time Integral (VTI) as well as changes in heart rate (HR) and mean arterial blood pressure (MABP), white blood cell count at day 2-4 and C-reactive protein (CRP) at day 2-4 were evaluated as indicators of neuromodulation and physiological effect of the treatment.

A total of thirteen patients were included in the study. Baseline characteristics are summarized in Supplementary Table 1.

Participants' splenic arterial anatomy was assessed using computed tomography angiography (CTA) for the determination of arterial lumen diameter to determine the cuff size to be used during study procedure and to confirm anatomical characteristics of the splenic neurovascular bundle. All participants underwent Minimal Invasive Esophagectomy with intrathoracic anastomosis (MIE) and were planned to undergo the experimental procedure. During the MIE-IL procedure, the splenic NVB was isolated, and the investigational lead was implanted laparoscopically around the NVB (2A). The stimulation lead consisting of the lead body, with a distal end cuff electrode and a proximal connector, was connected via an extension lead to an External Pulse Generator (EPG) (schematic presentation 2B). Thereafter the ultrasound transducer was introduced into the abdomen and placed on the NVB distal of the lead cuff to visualize SpA BF during stimulation.

In the abdominal phase of this procedure, the splenic artery is partially exposed due to the lymphadenectomy that is routinely performed. The splenic artery including the neurovascular bundle (NVB) was further mobilized to allow placement of the cuff electrode. Following mobilization, the lead was introduced through a trocar into the abdomen and positioned around the splenic NVB. The other end of the lead was connected to the EPG via the extension cable. A single impedance measurement was taken to ensure proper function of the lead. In case of high impedance (i.e. ≥2.5), the cuff electrode was repositioned or replaced with a second lead. The ultrasound transducer was introduced in the abdominal cavity through one of the trocards, to collect velocity profiles after confirmation of cuff electrode function. The intraoperative stimulation was performed according to the predefined stimulation scheme. Total experimental procedure time (including additional dissection, lead placement, optimization of Doppler imaging, stimulation and lead removal) did not exceed 40 minutes.

The NVB was stimulated using parameters selected to cause a change in SpA BF, based on pig studies (Donega et al; PNAS 2021) as surrogate marker of NVB activation, and in silico modelling of human SpA NVB tissue (Gupta et al, Commun Biol. 2020; 3:577). The parameters chosen were 400 μs pulse width, biphasic, frequency of 10 Hz, 60 seconds duration. The amplitude was adjusted over the course of the study based on experience with prior study participants in relation to target engagement and safety. The number of three stimulations were based on studies performed in an acute LPS-induced inflammation model (Guyot et al, BBI 2019). The NVB was stimulated in the majority of participants three times, the starting amplitude of 10 mA (4 μC) was selected as the minimal amplitude to give a change in the SpA BF envisaged in this study.

All stimulations consisted of a biphasic pulse with 10 Hz and 1-minute duration. Stimulation amplitudes varied to investigate the effect of step-up stimulation. Participants were expected to be stimulated 1 to 3 times during the course of the procedure. Impedance measurements were taken before each stimulation to confirm cuff function. After each stimulation, splenic blood flow and cardiovascular metrics had to return to baseline before instigating a new stimulation. After the last stimulation, a final impedance measurement was taken before the lead is removed from the splenic NVB and abdominal cavity.

Splenic artery blood velocity measurements (PSV, EDV and VTI) were obtained using pulse wave Doppler. A laparoscopic ultrasound transducer (L51K, Hitachi Medical Systems B.V., Reeuwijk, the Netherlands) was placed distally from the cuff on the splenic NVB. The pulse wave Doppler signal was optimized intraoperatively by a trained investigator (DB) using beam steering and angle correction. PSV, EDV and VTI were recorded as still frame flow velocity profiles. Two baseline recordings were captured prior to each stimulation and during each stimulation (after 15 and 40 seconds) two recordings were captured.

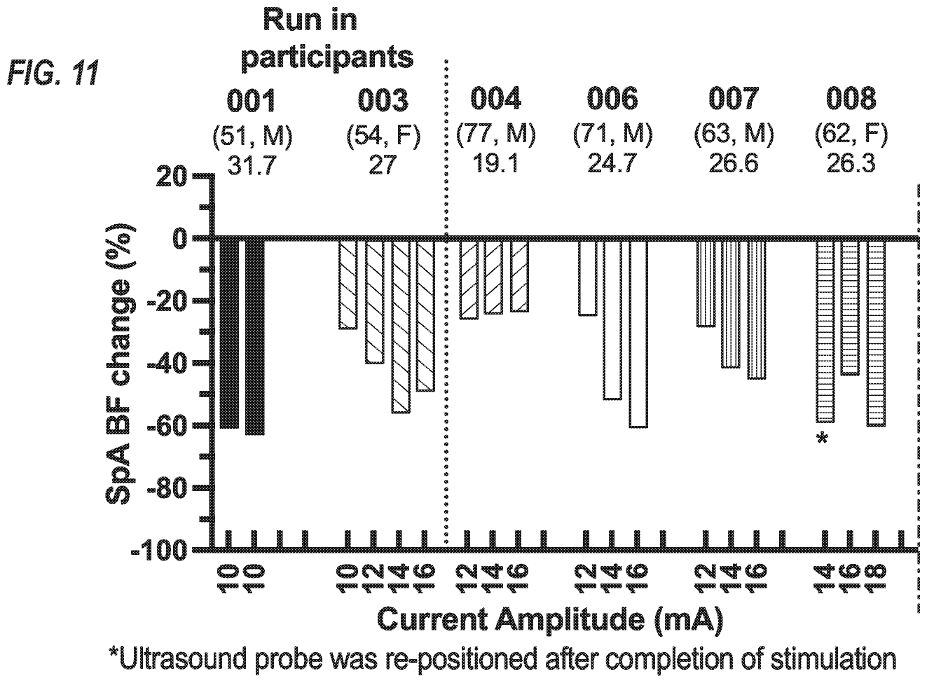

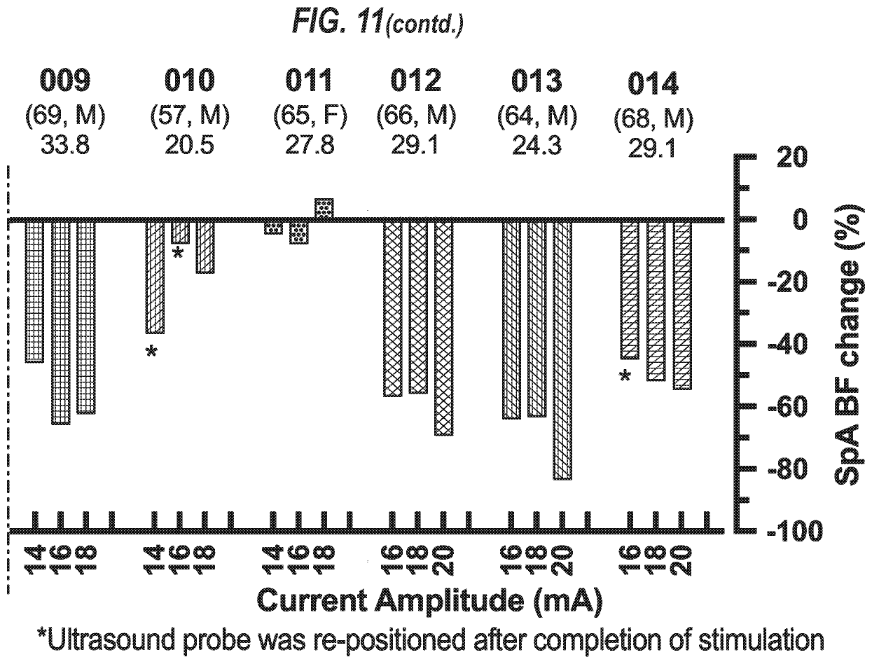

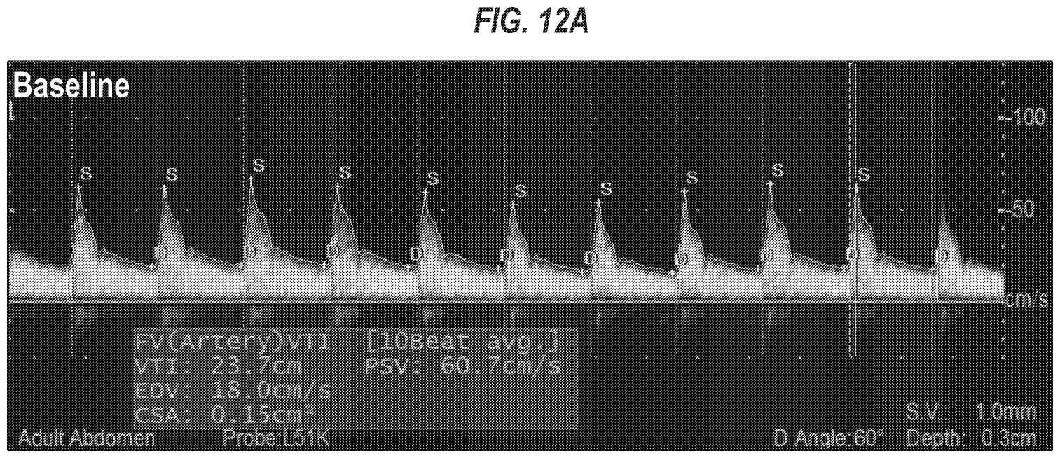

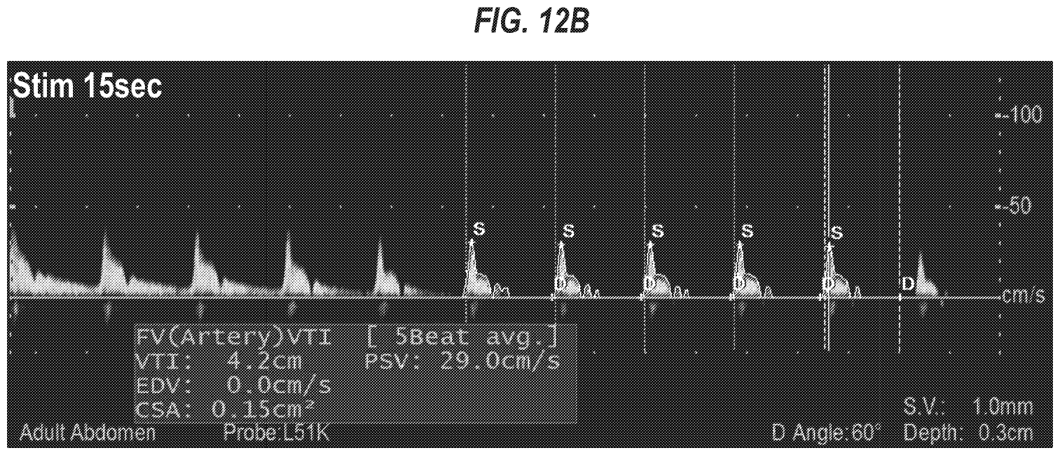

The lead-cuff was successfully applied to and removed from the NVB in all participants. Continuous current intensities between 10 and 20 mA were applied which resulted in consistent and reproducible decrease in SpA BF, as represented by VTI, in 97% of the participants confirming target engagement, as illustrated in FIGS. 11, 12, and 13.

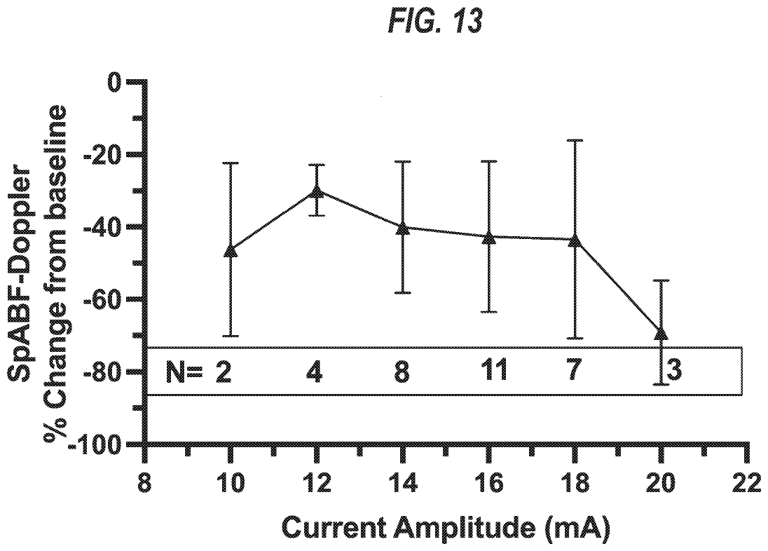

The blood velocity metrices PSV and the EDV demonstrated similar results. A correlation was observed between the amplitude and the BF change, as illustrated in FIG. 13. Impedance measurements taken after stimulation and prior to cuff removal confirmed that the lead was electrically functional in all patients at end of the investigational procedure, all measurements were within acceptable level of below 2.5 kΩ ranging from 0.3 kΩ to 1.5 kΩ.

Blood pressure (MABP) was continuously monitored using a patient monitoring system during the surgical procedure as is standard care. In parallel with the blood velocity measurements, two baseline recordings were manually documented from the system as well as during each stimulation.

CRP measurements and leukocyte count at postoperative day 2, 3 and 4 were assessed as part of patient care. CRP levels were determined in the morning (at 08.00 am) by immunoturbidimetric assay (Roche/Hitachi cobas C system, Roche).

The results from this thirteen patients' study were compared in a post-hoc analysis to patients who in 2019-2020 had undergone esophagectomy in the same hospital with the same pre- and post-operative treatment. In these patients, CRP levels were collected as part of the clinical postoperative care (day 2-4) since CRP is a marker for postoperative complications. To better compare groups, a Propensity Score Matched (PSM) analysis on postoperative complications was performed, matching for complications needing intervention (Clavien P A, Ann Surg. 2009), pneumonia and anastomotic leakage.

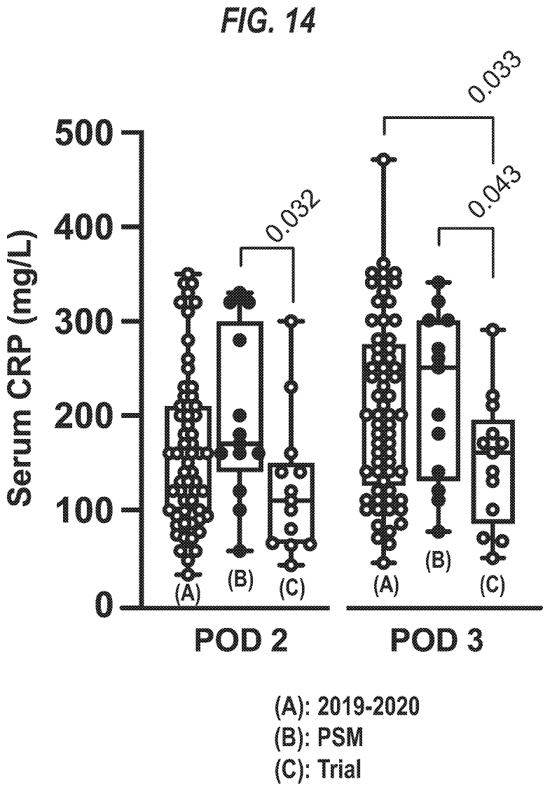

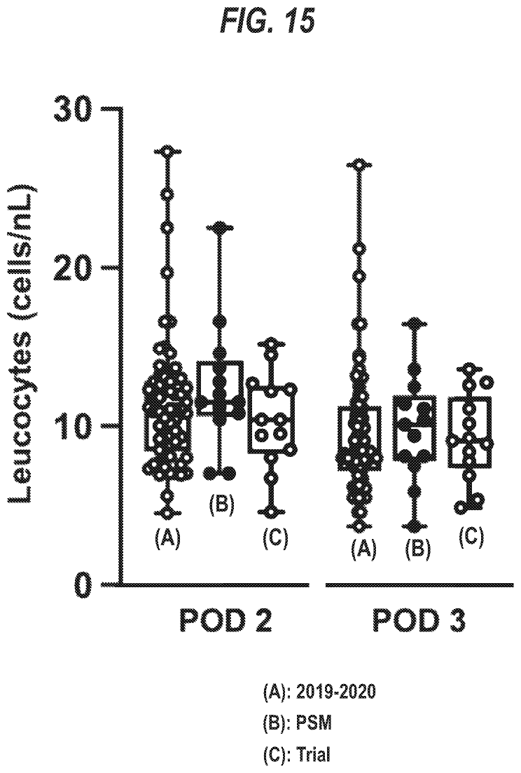

CRP values at day 2 and 3 were significantly reduced in the patients that underwent intra-operative splenic NVB stimulation (“Trial”) compared to the PSM control group (124.2±20.3 versus 196.7±24.7 respectively on day 2 (p=0.032); 150.5±19.0 versus 220.5±24.4, respectively on day 3 (p=0.033,)), as illustrated in FIG. 14. Specifically, FIG. 14 shows how splenic arterial (SpA) neurovascular bundle (NVB) stimulation in patients undergoing minimal invasive esophagectomy-Ivor Lewis (MIE-LI) decreases C-reaction protein levels on postoperative day (POD) 2 and POD 3 between patients that underwent SpA NVB stimulation (n=13) [TRIAL], a Prosperity Score Matched (PSM) control group (n=13) [PSM]; and patients (n=65) that underwent MIE-LI in the same hospital in 2019-2020 receiving the same surgery and post-operative care [2019-2020]. As illustrated in FIG. 14, when comparing the Study patients to the total 2019-2020 cohort (n=65) the CRP levels were reduced but did not reach significance on day 2 (124.2±20.3 versus 165.3±10.6, respectively (p=0.077)), but reached significance on day 3 (150.5±19.0 versus 206.7=11.6, respectively (p=0.043)). On day 4 after surgery, no significant differences in CRP levels between the cohorts were observed (Trial vs PSM p=0.413; Trial vs 2019-2020 p=0.323). The number of leucocytes, the cells mainly responsible for the production of inflammatory mediators, did not show a difference between groups, as illustrated in FIG. 15, implying that stimulation had a direct effect on the inflammatory process or; in line with what is expected based in the experimentally found underlying mechanism. Specifically, FIG. 15 shows how splenic arterial (SpA) neurovascular bundle (NVB) stimulation in patients undergoing minimal invasive esophagectomy-Ivor Lewis (MIE-LI) does not affect leukocytes counts in the blood on postoperative day (POD) 2 and POD 3 between patients that underwent SpA NVB stimulation (n=13) [TRIAL], a Prosperity Score Matched (PSM) control group (n=13) [PSM]; and patients that underwent MIE-LI in the same hospital in 2019-2020 receiving the same surgery and post-operative care [2019-2020]. In summary, CRP levels in the trial patients were respectively reduced to 124.2 and 150.5 mg/L on day 2 and 3 post-surgery, providing evidence that neuromodulation via the splenic nerve can result in reduction of the post-operative complication rate.

Further support for this conclusion comes from a few studies, which have provided insight in the interpretation of CRP levels after major abdominal surgery (Straatman et al. 2015, Adamina et al. 2014, Platt J J et al. 2012). For example, postoperative CRP levels have been shown to correlate with complications in patients undergoing open versus laparoscopic colorectal surgery regardless of surgical approach (Straatman Surgical 2018). Moreover, CRP levels on the third postoperative day after major abdominal surgery were found to be predictive for major complications (Straatman et al. 2015). Several cut-offs for CRP were proposed; a two-cut-off system may be applied, consisting of a safe discharge criterion with CRP levels below 75 mg/L. A second cut-off was set at 215 mg/L (probability 20%) to serve as a predictor of complications. The foregoing data was based on several major abdominal surgeries other than MIE-IL.

The clinical trial presented here is the first-in-human trial showing that splenic neuromodulation could be beneficial for the reduction/prevention of major complications after MIE-IL or other elective surgeries, improving the outcome for patients in relation to post-operative complications. A schematic representation of the proposed mechanism of action is shown in FIG. 6. Moreover, this clinical study is demonstrating the safety and feasibility of applying a cuff using a laparoscopic procedure and showing that results in target nerve engagement using BF as surrogate biomarker. All together this opens the way for further development of SpA NVB stimulation for chronic inflammatory diseases.

No adverse events experienced by patients in this study were attributed to placement or removal of the cuff nor were they attributed to the stimulation. Cardiovascular endpoints were continuously monitored during the study procedure, although some changes in heart rate and mean arterial blood pressure were observed, no clinically significant observations were made.

SPSS version 26.0 (IBM, Armonk, NY) was used to perform statistical analyses. No sample size calculation was performed for the clinical study. After the first three patients (deemed the “run in period”), the number of ten participants was deemed sufficient whether cuff electrode placement and removal was safe and feasible. A logistic regression analysis was performed on postoperative occurrence of any Clavien Dindo complication ≥3, pneumonia or anastomotic leakage to use resulting propensity variables to select controls for the trial patients.

Graphs were made with Prism 8.3 (GraphPad Software, La Jolla, CA, USA).

SUPPLEMENTARY TABLE 1
Baseline characteristics of all patients that underwent esophagectomy
in 2019-2020 (excluding trial participants), the participants
in the pilot trial and a complication matched cohort from the
2019-2020 group. Values are shown as continuous (SD or IQR)
or number (%). Comparisons were made using the t test, Mann
Whitney U test or Chi square test as appropriate.

| | | | Matched | | SpA NVB | |
|---|---|---|---|---|---|---|
| | 2019-2020 | | cohort | | stimulation | |
| | (n = 65) | | (n = 13) | | (n = 13) | |
| Age, years (SD) | 63.9 | (9.6) | 65.2 | (13.0) | 63.0 | (7.0) |
| Sex, male (%) | 54 | (83) | 9 | (70) | 9 | (70) |
| BMI, kg/m² (SD) | 27.2 | (4.7) | 25.6 | (4.1) | 26.0 | (4.8) |
| Preoperative status, | 17 | (27) | 2 | (15) | 3 | (23) |
| ASA ≥3 (%) | | | | | | |
| Comorbidity (%) | 44 | (68) | 7 | (54) | 6 | (54) |
| Cardiovascular (%) | 33 | (51) | 7 | (54) | 3 | (23) |
| Pulmonary (%) | 9 | (14) | 1 | (8) | 3 | (23) |
| Diabetes (%) | 9 | (14) | 0 | (0) | 2 | (15) |
| Pathological tumor | 28 | (44) | 4 | (31) | 7 | (54) |
| stage, ≥3 (%) | | | | | | |
| Surgery duration, min (SD) | 248 | (36) | 243 | (34) | 262 | (34) |

| | | | |
|---|---|---|---|
| Blood loss, mL [IQR] | 100 [50- | 150 [50- | 150 [100- |
| | 150] | 250] | 200] |

It is to be understood that the invention is not limited in its application to the details of construction and the arrangement of the components set forth herein. The invention is capable of other embodiments and of being practiced or being carried out in various ways. Variations and modifications of the foregoing are within the scope of the present invention. It should be understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

While the preferred embodiments of the invention have been shown and described, it will be apparent to those skilled in the art that changes and modifications may be made therein without departing from the spirit of the invention, the scope of which is defined by this description.

### Exemplary Embodiments

A system for stimulating the spleen and/or neural activity of one or more nerves supplying the spleen for reducing post-operative surgical complications, the system comprising: at least one transducer in signaling contact with the spleen and/or one or more nerves supplying the spleen; and at least one controller coupled to the at least one transducer, the at least one controller configured to control the operation of the at least one transducer to apply a signal to the spleen and/or the one or more nerves supplying the spleen, wherein the signal produces a reduction in C-reactive protein (CRP) levels resulting in reduced post-operative surgical complications.

A system for stimulating neural activity of one or more nerves supplying the spleen, wherein the one or more nerves directly innervates the spleen, for reducing post-operative surgical complications, the system comprising: at least one transducer in signaling contact with one or more nerves supplying the spleen; and at least one controller coupled to the at least one transducer, the at least one controller configured to control the operation of the at least one transducer to apply a signal to the spleen or the one or more nerves supplying the spleen, wherein the signal produces a reduction in C-reactive protein (CRP) levels resulting in reduced post-operative surgical complications.

A system for stimulating the neural activity of the spleen and/or one or more nerves supplying the spleen for reducing post-operative surgical complications, the system comprising: at least one transducer in signaling contact with the spleen and/or one or more nerves innervating the spleen; and at least one controller electrically coupled to the at least one transducer, the at least one controller configured to control the operation of the at least one transducer to apply a signal to the spleen and/or one or more nerves, wherein the signal produces a reduction in C-reactive protein (CRP) levels resulting in reduced post-operative surgical complications.

A system for stimulating the neural activity of one or more nerves supplying the spleen for reducing post-operative surgical complications, wherein the one or more nerves innervates the spleen, the system comprising: at least one transducer in signaling contact with the one or more nerves innervating the spleen; and at least one controller electrically coupled to the at least one transducer, the at least one controller configured to control the operation of the at least one transducer to apply a signal to the one or more nerves, wherein the signal produces a reduction in C-reactive protein (CRP) levels resulting in reducing post-operative surgical complications.

A system for reducing post-operative surgical complications by eliciting a reduction in an inflammatory parameter, the system comprising: at least one transducer in signaling contact with a target selected from at least one nerve supplying the spleen and a spleen, wherein the transducer is configured to be in temporary signaling contact with the target; and at least one controller coupled to the at least one transducer, the at least one controller configured to control the operation of the at least one transducer to apply a signal to the target, wherein temporary application of the signal to the target results in a reduction of post-operative surgical complications and elicits a reduction in an inflammatory parameter selected from at least one of C-reactive protein (CRP) levels and Interleukin-6 (IL-6) levels.

A system for stimulating the spleen and/or neural activity of one or more nerves supplying the spleen, the system comprising: at least one transducer in signaling contact with the spleen and/or one or more nerves supplying the spleen; and at least one controller coupled to the at least one transducer, the at least one controller configured to control the operation of the at least one transducer to apply a signal to the spleen and/or the one or more nerves supplying the spleen, wherein the signal produces a reduction in C-reactive protein (CRP) levels.

A system for stimulating neural activity of one or more nerves supplying the spleen, wherein the one or more nerves directly innervates the spleen, the system comprising: at least one transducer in signaling contact with one or more nerves supplying the spleen; and at least one controller coupled to the at least one transducer, the at least one controller configured to control the operation of the at least one transducer to apply a signal to the spleen or the one or more nerves supplying the spleen, wherein the signal produces a reduction in C-reactive protein (CRP) levels.

A system for stimulating the neural activity of the spleen and/or one or more nerves supplying the spleen, the system comprising: at least one transducer in signaling contact with the spleen and/or one or more nerves innervating the spleen; and at least one controller electrically coupled to the at least one transducer, the at least one controller configured to control the operation of the at least one transducer to apply a signal to the spleen and/or one or more nerves, wherein the signal produces a reduction in C-reactive protein (CRP) levels.

A system for stimulating the neural activity of one or more nerves supplying the spleen, wherein the one or more nerves innervates the spleen, the system comprising: at least one transducer in signaling contact with the one or more nerves innervating the spleen; and at least one controller electrically coupled to the at least one transducer, the at least one controller configured to control the operation of the at least one transducer to apply a signal to the one or more nerves, wherein the signal produces a reduction in C-reactive protein (CRP) levels.

A system for eliciting a reduction in an inflammatory parameter, the system comprising: at least one transducer in signaling contact with a target selected from at least one nerve supplying the spleen and a spleen, wherein the transducer is configured to be in temporary signaling contact with the target; and at least one controller coupled to the at least one transducer, the at least one controller configured to control the operation of the at least one transducer to apply a signal to the target, wherein temporary application of the signal to the target elicits a reduction in an inflammatory parameter selected from at least one of C-reactive protein (CRP) levels and Interleukin-6 (IL-6) levels

A method for stimulating the spleen and/or neural activity of one or more nerves supplying the spleen of a subject for reducing post-operative surgical complications, the method comprising: providing a system of any one of the preceding systems; positioning at least one transducer in signaling contact with the spleen and/or one or more nerves supplying the spleen; and controlling the operation of the at least one transducer with at least one controller coupled to the at least one transducer, the at least one controller configured to control the operation of the at least one transducer to apply a signal to the spleen and/or the one or more nerves supplying the spleen, wherein the signal produces a reduction in C-reactive protein (CRP) levels resulting in reduced post-operative surgical complications.

A method for stimulating neural activity of one or more nerves supplying the spleen of a subject, wherein the one or more nerves directly innervates the spleen, for reducing post-operative surgical complications, the method comprising: providing a system of any one of the preceding systems; positioning at least one transducer in signaling contact with one or more nerves supplying the spleen; and controlling the operation of the at least one transducer with at least one controller coupled to the at least one transducer, the at least one controller configured to control the operation of the at least one transducer to apply a signal to the spleen or the one or more nerves supplying the spleen, wherein the signal produces a reduction in C-reactive protein (CRP) levels resulting in reduced post-operative surgical complications.

A method for stimulating the neural activity of the spleen and/or one or more nerves supplying the spleen of a subject for reducing post-operative surgical complications, the method comprising: providing a system of any one of the preceding systems; positioning at least one transducer in signaling contact with the spleen and/or one or more nerves innervating the spleen; and controlling the operation of the at least one transducer with at least one controller electrically coupled to the at least one transducer, the at least one controller configured to control the operation of the at least one transducer to apply a signal to the spleen and/or one or more nerves, wherein the signal produces a reduction in C-reactive protein (CRP) levels resulting in reduced post-operative surgical complications.

A method for stimulating the neural activity of one or more nerves supplying the spleen of a subject for reducing post-operative surgical complications, wherein the one or more nerves innervates the spleen, the method comprising: providing a system of any one of the preceding systems; positioning at least one transducer in signaling contact with the one or more nerves innervating the spleen; and controlling the operation of the at least one transducer with at least one controller electrically coupled to the at least one transducer, the at least one controller configured to control the operation of the at least one transducer to apply a signal to the one or more nerves, wherein the signal produces a reduction in C-reactive protein (CRP) levels resulting in reducing post-operative surgical complications.

A method for reducing post-operative surgical complications by eliciting a reduction in an inflammatory parameter, the method comprising: providing a system of any one of the preceding systems; positioning at least one transducer in signaling contact with a target selected from at least one nerve supplying the spleen and a spleen, wherein the transducer is configured to be in temporary signaling contact with the target; and controlling the operation of the at least one transducer with at least one controller coupled to the at least one transducer, the at least one controller configured to control the operation of the at least one transducer to apply a signal to the target, wherein temporary application of the signal to the target results in a reduction of post-operative surgical complications and elicits a reduction in an inflammatory parameter selected from at least one of C-reactive protein (CRP) levels and Interleukin-6 (IL-6) levels.

A method of reversibly stimulating neural activity in the spleen and/or one or more nerves supplying the spleen of a subject, wherein the one or more nerves innervates the spleen, the method comprising: providing a system of any one of the preceding systems; positioning at least one transducer in signaling contact with the spleen and/or one or more nerves; and controlling the operation of the at least one transducer with at least one controller to apply a signal to the one or more nerves to stimulate neural activity, wherein the signal produces a reduction in C-reactive protein (CRP) levels resulting in reducing post-operative surgical complications.

Any of the preceding systems or methods, wherein the signal further produces a change in a physiological parameter indicative of target engagement.

Any of the preceding systems or methods, wherein CRP and/or IL-6 is measured in a biological sample selected from the group consisting of blood, serum and plasma.

Any of the preceding systems or methods, wherein the transducer is signaling contact with the target for a contact time period no longer than 24 hours, 18 hours, 15 hours, 12 hours, 10 hours, 9 hours, 8 hours, 7 hours, 6 hours, 5 hours, 4 hours, 3 hours, 2 hours, 1 hour, 30 minutes, or 20 minutes.

Any of the preceding systems or methods, wherein transducer is in signaling contact with the target on a continuous or intermittent basis during the contact time period.

Any of the preceding systems or methods, wherein the application of the signal is continuous or intermittent during the contact time period.

Any of the preceding systems or methods, wherein the reduction in the inflammatory parameter is detectable within 1 day, 2 days, 3 days, 4 days, or 5 days from the application of the signal.

Any of the preceding systems or methods, wherein the reduction in the inflammatory parameter is detectable within 1 day, 2 days, 3 days, 4 days, or 5 days following a first application of the signal.

Any of the preceding systems or methods, wherein the reduction in CRP levels in plasma is to less than 245 mg/L.

Any of the preceding systems or methods, wherein the reduction in CRP levels in plasma is to less than 215 mg/L.

Any of the preceding systems or methods, wherein the transducer comprises a neural interface with at least one electrode.

Any of the preceding systems or methods, wherein the neural interface is placed around at least one splenic nerve.

Any of the preceding systems or methods, wherein the neural interface is placed around at least one splenic arterial nerve.

Any of the preceding systems or methods, wherein the neural interface is placed around a neurovascular bundle.

Any of the preceding systems or methods, wherein the neural interface is placed on at least one splenic nerve.

Any of the preceding systems or methods, wherein the neural interface is placed on at least one splenic arterial nerve.

Any of the preceding systems or methods, wherein the neural interface is configured to be placed on the splenic artery.

Any of the preceding systems or methods, wherein the neural interface is placed at least partially inside the splenic artery.

Any of the preceding systems or methods, wherein the neural interface comprises: two electrically active electrode arms; an inert arm located in between each of the two electrically active electrode arms; and a spine that provides mechanical and electrical connection between the two electrically active electrode arms and the inert arm.

Any of the preceding systems or methods, wherein the signal applied by the transducer is an electrical signal.

Any of the preceding systems or methods, wherein the neural interface is placed in signaling contact with the spleen or nerve for less than one of the following of: ≤24 hours, ≤18 hours, ≤15 hours, or ≤12 hours.

Any of the preceding systems or methods, wherein the neural interface is placed in signaling contact with the spleen or nerve ≤seven days, wherein a day is a 24-hour period.

Any of the preceding systems or methods, wherein the transducer is an ultrasound transducer.

Any of the preceding systems or methods, wherein the transducer is in physical contact with the spleen and/or the one or more nerves.

Any of the preceding systems or methods, wherein the change in a physiological parameter indicative of the target engagement of the one or more target nerves results in a decrease in Interleukin-6 (IL-6) levels.

Any of the preceding systems or methods, wherein the electrical signal comprises a plurality of pulses, wherein the pulses are biphasic and bipolar pulses.

Any of the preceding systems or methods, wherein the plurality of pulses comprise pulse width is ≥0.1 ms and ≤5 ms.

Any of the preceding systems or methods, wherein the electrical signal has a frequency of 0.1 Hz to ≤300 Hz.

Any of the preceding systems or methods, wherein the electrical signal has a charge density of 5 μC per cm² per phase to 250 μC per cm² per phase. Any of the preceding systems or methods, wherein the electrical signal has a charge density of ≥40 μC per cm² per phase to ≤260 μC per cm² per phase. Any of the preceding systems or methods, wherein the electrical signal has a charge density of ≥70 μC per cm² per phase to ≤300 μC per cm² per phase.

Any of the preceding systems or methods, wherein the nerve is associated with a splenic nerve.

Any of the preceding systems or methods, wherein the transducer is extravascular.

Any of the preceding systems or methods, wherein the nerve is associated with a splenic arterial nerve.

Any of the preceding systems or methods, wherein the transducer is an intravascular neural interface with the splenic arterial nerve.

1.-79. (canceled)

80. A system for stimulating neural activity of one or more nerves supplying a spleen for reducing post-operative surgical complications, wherein the one or more nerves innervates the spleen, the system comprising:
at least one transducer in signaling contact with the one or more nerves innervating the spleen; and
at least one controller electrically coupled to the at least one transducer, the at least one controller configured to control operation of the at least one transducer to apply an electrical signal to the one or more nerves, wherein the signal produces a reduction in C-reactive protein (CRP) levels resulting in reducing post-operative surgical complications.

81. The system of claim 80, wherein the reduction in CRP levels in plasma is to less than 245 mg/L.

82. The system of claim 80, wherein the reduction in CRP levels in plasma is to less than 215 mg/L.

83. The system of claim 80, wherein the transducer comprises a neural interface with at least one electrode.

84. The system of claim 83, wherein the neural interface is configured to be placed around at least one splenic nerve.

85. The system of claim 83, wherein the neural interface is configured to be placed around at least one splenic arterial nerve.

86. The system of claim 83, wherein the neural interface is configured to be placed around a neurovascular bundle.

87. The system of claim 83, wherein the neural interface is configured to be placed on at least one splenic nerve.

88. The system of claim 83, wherein the neural interface is configured to be placed on at least one splenic arterial nerve.

89. The system of claim 83, wherein the neural interface is configured to be placed on the splenic artery.

90. The system of claim 83, wherein the neural interface is placed at least partially inside the splenic artery.

91. The system of claim 83, wherein the neural interface comprises: two electrically active electrode arms; an inert arm located in between each of the two electrically active electrode arms; and a spine that provides mechanical and electrical connection between the two electrically active electrode arms and the inert arm.

92. The system of claim 83, wherein the neural interface is placed in signaling contact with the spleen or nerve for less than one of the following of: ≤24 hours, ≤18 hours, ≤15 hours, or ≤12 hours.

93. The system of claim 80, wherein the electrical signal has a charge density of ≥40 μC per cm² per phase to ≤260 μC per cm².

94. A system for reducing post-operative surgical complications by eliciting a reduction in an inflammatory parameter, the system comprising:
at least one transducer in signaling contact with a target selected from at least one nerve supplying a spleen and the spleen, wherein the transducer is configured to be in temporary signaling contact with the target; and
at least one controller coupled to the at least one transducer, the at least one controller configured to control operation of the at least one transducer to apply a signal to the target, wherein temporary application of the signal to the target results in a reduction of post-operative surgical complications and elicits a reduction in an inflammatory parameter selected from at least one of C-reactive protein (CRP) levels and Interleukin-6 (IL-6) levels.

95. The system of claim 94, wherein the signal further produces a change in a physiological parameter indicative of target engagement.

96. The system of claim 94, wherein CRP and/or IL-6 is measured in a biological sample selected from the group consisting of blood, serum, and plasma.

97. The system of claim 94, wherein the transducer is in signaling contact with the target for a contact time period no longer than 24 hours, 18 hours, 15 hours, 12 hours, 10 hours, 9 hours, 8 hours, 7 hours, 6 hours, 5 hours, 4 hours, 3 hour, 2 hours, 1 hour, 30 minutes, or 20 minutes.

98. The system of claim 94, wherein the reduction in the inflammatory parameter is detectable within 1 day, 2 days, 3 days, 4 days, or 5 days following a first application of the signal.

99. The system of claim 94, wherein the reduction in an inflammatory parameter is a decrease in Interleukin-6 (IL-6) levels.

100. A method for stimulating neural activity of one or more nerves supplying a spleen of a subject, wherein the one or more nerves directly innervates the spleen, for reducing post-operative surgical complications, the method comprising:
providing a system of claim 80;
positioning at least one transducer in signaling contact with one or more nerves supplying the spleen; and
controlling operation of the at least one transducer with at least one controller coupled to the at least one transducer, the at least one controller configured to control the operation of the at least one transducer to apply an electrical signal to the spleen or the one or more nerves supplying the spleen, wherein the electrical signal produces a reduction in C-reactive protein (CRP) levels resulting in reduced post-operative surgical complications.

101. The method of claim 100, wherein the reduction in CRP levels in plasma is to less than 245 mg/L.

102. The method of claim 100, wherein the reduction in CRP levels in plasma is to less than 215 mg/L.

103. The method of claim 100, wherein the transducer comprises a neural interface, the neural interface comprising: two electrically active electrode arms; an inert arm located in between each of the two electrically active electrode arms; and a spine that provides mechanical and electrical connection between the two electrically active electrode arms and the inert arm.

104. The method of claim 100, wherein the electrical signal has a charge density of ≥40 μC per cm² per phase to ≤260 μC per cm² per phase.
